SEQUENCE LISTING (20 sequences)

SEQ ID NO: 1 - protein, 234 aa, Artificial
  HyHEL10 scFv peptide
DIVLTQSPATLSVTPGNSVSLSCRASQSIGNNLHWYQQKSHESPRLLIKYASQSISGIPSRFSGSGSGTDFTLSINSVETEDFGMYFCQQSNSWPYTFGGGTKLEIGGGGSGGGGSGGGGSDIQLQESGPSLVKPSQTLSLTCSVTGDSITSDYWSWIRKFPGNRLEYMGYVSYSGSTYYNPSLKSRISITRDTSKNQYYLDLNSVTTEDTATYYCANWDGDYWGQGTLVTVSA

SEQ ID NO: 2 - DNA, 702 bp, Artificial
  DNA sequence coding HeyHEL10 scFv peptide
gatatcgtcctgacccagagcccggcgaccctctcggtcacccccggcaactcggtgtccctctcgtgccgcgcctcgcagtcgatcggcaacaacctccactggtatcagcagaagtcgcacgagagcccgcgcctcctgatcaagtacgccagccagtcgatctcgggaatcccgtcgcgcttcagcggctcgggctcgggcaccgacttcaccctgtcgatcaacagcgtcgagacggaggacttcggcatgtacttctgccagcagtcgaacagctggccgtacaccttcggcggcggtaccaagctggagatcggcgggggcggtagcggcgggggcggtagcggcgggggcggtagcgatatccagctgcaggagtcgggcccgagcctcgtcaagccgtcgcagaccctgtcgctcacctgcagcgtcaccggcgactcgatcacctcggactactggtcgtggattcgcaagttccccggcaaccgcctcgagtacatgggctacgtcagctactcgggcagcacctactacaacccctcgctgaagagccgcatctcgatcacccgcgacacctccaagaaccagtactacctggacctcaactcggtcaccaccgaggacaccgccacctactactgcgcgaactgggacggcgactactggggccagggcaccctcgtcaccgtctccgcg

SEQ ID NO: 3 - protein, 10 aa, Artificial
  poly asparatic acid
DDDDDDDDDD

SEQ ID NO: 4 - DNA, 30 bp, Artificial
  DNA sequence coding poly asparatic acid
gatgatgatgacgatgacgatgatgacgat

SEQ ID NO: 5 - protein, 5 aa, Artificial
  Linker peptide
GGGGS

SEQ ID NO: 6 - DNA, 15 bp, Artificial
  DNA sequence coding the Linker peptide
ggcgggggcggtagc

SEQ ID NO: 7 - protein, 404 aa, Artificial
  HeyHEL10 scFv peptide labeled with poly asparatic acid
DIVLTQSPATLSVTPGNSVSLSCRASQSIGNNLHWYQQKSHESPRLLIKYASQSISGIPSRFSGSGSGTDFTLSINSVETEDFGMYFCQQSNSWPYTFGGGTKLEIGGGGSGGGGSGGGGSDIQLQESGPSLVKPSQTLSLTCSVTGDSITSDYWSWIRKFPGNRLEYMGYVSYSGSTYYNPSLKSRISITRDTSKNQYYLDLNSVTTEDTATYYCANWDGDYWGQGTLVTVSAGGGGSGGGGSDDDDDDDDDDDDDDDDDDDDDDDDDDDDDDDDDDDDDDDDGGGGSGGGGSGGGGSGGGGSDDDDDDDDDDDDDDDDDDDDDDDDDDDDDDDDDDDDDDDDGGGGSGGGGSGGGGSGGGGSDDDDDDDDDDDDDDDDDDDDDDDDDDDDDDDDDDDDDDDD

SEQ ID NO: 8 - DNA, 1212 bp, Artificial
  DNA sequence coding HeyHEL10 scFv peptide labeled with poly asparatic acid
gatatcgtcctgacccagagcccggcgaccctctcggtcacccccggcaactcggtgtccctctcgtgccgcgcctcgcagtcgatcggcaacaacctccactggtatcagcagaagtcgcacgagagcccgcgcctcctgatcaagtacgccagccagtcgatctcgggaatcccgtcgcgcttcagcggctcgggctcgggcaccgacttcaccctgtcgatcaacagcgtcgagacggaggacttcggcatgtacttctgccagcagtcgaacagctggccgtacaccttcggcggcggtaccaagctggagatcggcgggggcggtagcggcgggggcggtagcggcgggggcggtagcgatatccagctgcaggagtcgggcccgagcctcgtcaagccgtcgcagaccctgtcgctcacctgcagcgtcaccggcgactcgatcacctcggactactggtcgtggattcgcaagttccccggcaaccgcctcgagtacatgggctacgtcagctactcgggcagcacctactacaacccctcgctgaagagccgcatctcgatcacccgcgacacctccaagaaccagtactacctggacctcaactcggtcaccaccgaggacaccgccacctactactgcgcgaactgggacggcgactactggggccagggcaccctcgtcaccgtctccgcgggcgggggcggtagcggcgggggcggtagcgatgatgatgacgatgacgatgatgacgatgatgatgatgacgatgacgatgatgacgatgatgatgatgacgatgacgatgatgacgatgatgatgatgacgatgacgatgatgacgatggcgggggcggtagcggcgggggcggtagcggcgggggcggtagcggcgggggcggtagcgatgatgatgacgatgacgatgatgacgatgatgatgatgacgatgacgatgatgacgatgatgatgatgacgatgacgatgatgacgatgatgatgatgacgatgacgatgatgacgatggcgggggcggtagcggcgggggcggtagcggcgggggcggtagcggcgggggcggtagcgatgatgatgacgatgacgatgatgacgatgatgatgatgacgatgacgatgatgacgatgatgatgatgacgatgacgatgatgacgatgatgatgatgacgatgacgatgatgacgat

SEQ ID NO: 9 - protein, 239 aa, Artificial
  D1.3 scFv peptide
QVQLKESGPGLVAPSQSLSITCTVSGFSLTGYGVNWVRQPPGKGLEWLGMIWGDGNTDYNSALKSRLSISKDNSKSQVFLKMNSLHTDDTARYYCARERDYRLDYWGQGTTLTVSSGGGGSGGGGSGGGGSDIQMTQSPASLSASVGETVTITCRASGNIHNYLAWYQQKQGKSPQLLVYYTTTLADGVPSRFSGSGSGTQYSLKINSLQPEDFGSYYCQHFWSTPRTFGGGTKLEIKR

SEQ ID NO: 10 - DNA, 717 bp, Artificial
  DNA sequence coding D1.3 scFv peptide
caggtgcagctgaaggagtcaggacctggcctggtggcgccctcacagagcctgtccatcacatgcaccgtctcagggttctcattaaccggctatggtgtaaactgggttcgccagcctccaggaaagggtctggagtggctgggaatgatttggggtgatggaaacacagactataattcagctctcaaatccagactgagcatcagcaaggacaactccaagagccaagttttcttaaaaatgaacagtctgcacactgatgacacagccaggtactactgtgccagagagagagattataggcttgactactggggccaaggcaccactctcacagtctcctcaggcgggggcggtagcggcgggggcggtagcggcgggggcggtagcgacatccagatgactcagtctccagcctccctttctgcgtctgtgggagaaactgtcaccatcacatgtcgagcaagtgggaatattcacaattatttagcatggtatcagcagaaacagggaaaatctcctcagctcctggtctattatacaacaaccttagcagatggtgtgccatcaaggttcagtggcagtggatcaggaacacaatattctctcaagatcaacagcctgcagcctgaagattttgggagttattactgtcaacatttttggagtactcctcggacgttcggtggaggcaccaagctggaaatcaaacgg

SEQ ID NO: 11 - protein, 12 aa, Artificial
  SiO2 affinity peptide
RGRRRRLSCRLL

SEQ ID NO: 12 - DNA, 36 bp, Artificial
  DNA sequence coding SiO2 affinity peptide
cgtggccgtcgtcgccgtctgagctgccgtctgctg

SEQ ID NO: 13 - protein, 575 aa, Artificial
  HyHEL10 scFv fragment labeled with SiO2 affinity peptide
DIVLTQSPATLSVTPGNSVSLSCRASQSIGNNLHWYQQKSHESPRLLIKYASQSISGIPSRFSGSGSGTDFTLSINSVETEDFGMYFCQQSNSWPYTFGGGTKLEIGGGGSGGGGSGGGGSDIQLQESGPSLVKPSQTLSLTCSVTGDSITSDYWSWIRKFPGNRLEYMGYVSYSGSTYYNPSLKSRISITRDTSKNQYYLDLNSVTTEDTATYYCANWDGDYWGQGTLVTVSAGGGGSGGGGSGGGGSGGGGSRGRRRRLSCRLLRGRRRRLSCRLLRGRRRRLSCRLLRGRRRRLSCRLLRGRRRRLSCRLLRGRRRRLSCRLLRGRRRRLSCRLLGGGGSGGGGSGGGGSGGGGSRGRRRRLSCRLLRGRRRRLSCRLLRGRRRRLSCRLLRGRRRRLSCRLLRGRRRRLSCRLLRGRRRRLSCRLLRGRRRRLSCRLLGGGGSGGGGSGGGGSGGGGSGGGGSRGRRRRLSCRLLRGRRRRLSCRLLRGRRRRLSCRLLRGRRRRLSCRLLRGRRRRLSCRLLRGRRRRLSCRLLRGRRRRLSCRLLRGRRRRLSCRLLRGRRRRLSCRLL

SEQ ID NO: 14 - DNA, 1725 bp, Artificial
  DNA sequence coding HyHEL10 scFv peptide labeled with SiO2 affinity peptide
gatatcgtcctgacccagagcccggcgaccctctcggtcacccccggcaactcggtgtccctctcgtgccgcgcctcgcagtcgatcggcaacaacctccactggtatcagcagaagtcgcacgagagcccgcgcctcctgatcaagtacgccagccagtcgatctcgggaatcccgtcgcgcttcagcggctcgggctcgggcaccgacttcaccctgtcgatcaacagcgtcgagacggaggacttcggcatgtacttctgccagcagtcgaacagctggccgtacaccttcggcggcggtaccaagctggagatcggcgggggcggtagcggcgggggcggtagcggcgggggcggtagcgatatccagctgcaggagtcgggcccgagcctcgtcaagccgtcgcagaccctgtcgctcacctgcagcgtcaccggcgactcgatcacctcggactactggtcgtggattcgcaagttccccggcaaccgcctcgagtacatgggctacgtcagctactcgggcagcacctactacaacccctcgctgaagagccgcatctcgatcacccgcgacacctccaagaaccagtactacctggacctcaactcggtcaccaccgaggacaccgccacctactactgcgcgaactgggacggcgactactggggccagggcaccctcgtcaccgtctccgcgggcgggggcggtagcggcgggggcggtagcggcgggggcggtagcggcgggggcggtagccgtggccgtcgtcgccgtctgagctgccgtctgctgcgtggccgtcgtcgccgtctgagctgccgtctgctgcgtggccgtcgtcgccgtctgagctgccgtctgctgcgtggccgtcgtcgccgtctgagctgccgtctgctgcgtggccgtcgtcgccgtctgagctgccgtctgctgcgtggccgtcgtcgccgtctgagctgccgtctgctgcgtggccgtcgtcgccgtctgagctgccgtctgctgggcgggggcggtagcggcgggggcggtagcggcgggggcggtagcggcgggggcggtagccgtggccgtcgtcgccgtctgagctgccgtctgctgcgtggccgtcgtcgccgtctgagctgccgtctgctgcgtggccgtcgtcgccgtctgagctgccgtctgctgcgtggccgtcgtcgccgtctgagctgccgtctgctgcgtggccgtcgtcgccgtctgagctgccgtctgctgcgtggccgtcgtcgccgtctgagctgccgtctgctgcgtggccgtcgtcgccgtctgagctgccgtctgctgggcgggggcggtagcggcgggggcggtagcggcgggggcggtagcggcgggggcggtagcggcgggggcggtagccgtggccgtcgtcgccgtctgagctgccgtctgctgcgtggccgtcgtcgccgtctgagctgccgtctgctgcgtggccgtcgtcgccgtctgagctgccgtctgctgcgtggccgtcgtcgccgtctgagctgccgtctgctgcgtggccgtcgtcgccgtctgagctgccgtctgctgcgtggccgtcgtcgccgtctgagctgccgtctgctgcgtggccgtcgtcgccgtctgagctgccgtctgctgcgtggccgtcgtcgccgtctgagctgccgtctgctgcgtggccgtcgtcgccgtctgagctgccgtctgctg

SEQ ID NO: 15 - protein, 120 aa, Artificial
  10 repeats of SiO2 affinity peptide
RGRRRRLSCRLLRGRRRRLSCRLLRGRRRRLSCRLLRGRRRRLSCRLLRGRRRRLSCRLLRGRRRRLSCRLLRGRRRRLSCRLLRGRRRRLSCRLLRGRRRRLSCRLLRGRRRRLSCRLL

SEQ ID NO: 16 - DNA, 360 bp, Artificial
  DNA sequence coding 10 repeats of SiO2 affinity peptide
cgtggccgtcgtcgccgtctgagctgccgtctgctgcgtggccgtcgtcgccgtctgagctgccgtctgctgcgtggccgtcgtcgccgtctgagctgccgtctgctgcgtggccgtcgtcgccgtctgagctgccgtctgctgcgtggccgtcgtcgccgtctgagctgccgtctgctgcgtggccgtcgtcgccgtctgagctgccgtctgctgcgtggccgtcgtcgccgtctgagctgccgtctgctgcgtggccgtcgtcgccgtctgagctgccgtctgctgcgtggccgtcgtcgccgtctgagctgccgtctgctgcgtggccgtcgtcgccgtctgagctgccgtctgctg

SEQ ID NO: 17 - protein, 159 aa, Artificial
  storeptoadipic acid
DPSKDSKAQVSAAEAGITGTWYNQLGSTFIVTAGADGALTGTYESAVGNAESRYVLTGRYDSAPATDGSGTALGWTVAWKNNYRNAHSATTWSGQYVGGAEARINTQWLLTSGTTEANAWKSTLVGHDTFTKVKPSAASIDAAKKAGVNNGNPLDAVQQ

SEQ ID NO: 18 - DNA, 477 bp, Artificial
  gene for storeptoadipic acid
gacccctccaaggactcgaaggcccaggtctcggccgccgaggccggcatcaccggcacctggtacaaccagctcggctcgaccttcatcgtgaccgcgggcgccgacggcgccctgaccggaacctacgagtcggccgtcggcaacgccgagagccgctacgtcctgaccggtcgttacgacagcgccccggccaccgacggcagcggcaccgccctcggttggacggtggcctggaagaataactaccgcaacgcccactccgcgaccacgtggagcggccagtacgtcggcggcgccgaggcgaggatcaacacccagtggctgctgacctccggcaccaccgaggccaacgcctggaagtccacgctggtcggccacgacaccttcaccaaggtgaagccgtccgccgcctccatcgacgcggcgaagaaggccggcgtcaacaacggcaacccgctcgacgccgttcagcag

SEQ ID NO: 19 - protein, 408 aa, Artificial
  fused peptide aminoic acid
DIVLTQSPATLSVTPGNSVSLSCRASQSIGNNLHWYQQKSHESPRLLIKYASQSISGIPSRFSGSGSGTDFTLSINSVETEDFGMYFCQQSNSWPYTFGGGTKLEIGGGGSGGGGSGGGGSDIQLQESGPSLVKPSQTLSLTCSVTGDSITSDYWSWIRKFPGNRLEYMGYVSYSGSTYYNPSLKSRISITRDTSKNQYYLDLNSVTTEDTATYYCANWDGDYWGQGTLVTVSAGGGGSGGGGSGGGGSDPSKDSKAQVSAAEAGITGTWYNQLGSTFIVTAGADGALTGTYESAVGNAESRYVLTGRYDSAPATDGSGTALGWTVAWKNNYRNAHSATTWSGQYVGGAEARINTQWLLTSGTTEANAWKSTLVGHDTFTKVKPSAASIDAAKKAGVNNGNPLDAVQQ

SEQ ID NO: 20 - DNA, 1224 bp, Artificial
  gene for fused peptide
gatatcgtcctgacccagagcccggcgaccctctcggtcacccccggcaactcggtgtccctctcgtgccgcgcctcgcagtcgatcggcaacaacctccactggtatcagcagaagtcgcacgagagcccgcgcctcctgatcaagtacgccagccagtcgatctcgggaatcccgtcgcgcttcagcggctcgggctcgggcaccgacttcaccctgtcgatcaacagcgtcgagacggaggacttcggcatgtacttctgccagcagtcgaacagctggccgtacaccttcggcggcggtaccaagctggagatcggcgggggcggtagcggcgggggcggtagcggcgggggcggtagcgatatccagctgcaggagtcgggcccgagcctcgtcaagccgtcgcagaccctgtcgctcacctgcagcgtcaccggcgactcgatcacctcggactactggtcgtggattcgcaagttccccggcaaccgcctcgagtacatgggctacgtcagctactcgggcagcacctactacaacccctcgctgaagagccgcatctcgatcacccgcgacacctccaagaaccagtactacctggacctcaactcggtcaccaccgaggacaccgccacctactactgcgcgaactgggacggcgactactggggccagggcaccctcgtcaccgtctccgcgggcgggggcggtagcggcgggggcggtagcggcgggggcggtagcgacccctccaaggactcgaaggcccaggtctcggccgccgaggccggcatcaccggcacctggtacaaccagctcggctcgaccttcatcgtgaccgcgggcgccgacggcgccctgaccggaacctacgagtcggccgtcggcaacgccgagagccgctacgtcctgaccggtcgttacgacagcgccccggccaccgacggcagcggcaccgccctcggttggacggtggcctggaagaataactaccgcaacgcccactccgcgaccacgtggagcggccagtacgtcggcggcgccgaggcgaggatcaacacccagtggctgctgacctccggcaccaccgaggccaacgcctggaagtccacgctggtcggccacgacaccttcaccaaggtgaagccgtccgccgcctccatcgacgcggcgaagaaggccggcgtcaacaacggcaacccgctcgacgccgttcagcag

(19) United States
(12) Patent Application Publication
(10) Pub. No.: US 2008/0241964 A1
(43) Pub. Date: Oct. 2, 2008

(54) MATERIAL FOR IMPROVING SENSITIVITY OF MAGNETIC SENSOR AND METHOD THEREOF

(75) Inventors: Masaru Kaieda, Tokyo (JP); Takashi Ikeda, Yokohama-shi (JP); Kazumichi Nakahama, Tokyo (JP)

Correspondence Address: FITZPATRICK CELLA HARPER & SCINTO, 30 ROCKEFELLER PLAZA, NEW YORK, NY, 10112, US

(73) Assignee: CANON KABUSHIKI KAISHA, Tokyo (JP)

(21) Appl. No.: 12/054,231

(22) Filed: Mar. 24, 2008

(30) Foreign Application Priority Data
Mar. 27, 2007 (JP) 2007-081942
Mar. 7, 2008 (JP) 2008-057728

(51) Int. Cl. G01N 33/553; G01N 27/00

(52) U.S. Cl. 436/526; 422/82.01

(57) ABSTRACT

The present invention relates to a capture agent member being used in a magnetic sensor to detect the presence or concentration of a target substance in a test solution by detecting the presence or number of the magnetic marker, wherein the capture agent member contains a capture agent for capturing the target substance and a labeling agent serving as a nucleus for the magnetic marker to agglutinate, and wherein the capture agent is labeled with the labeling agent. The present invention relates to a material which improves detection sensitivity of a biosensor using a magnetic sensor while maintaining reactivity and dispersibility. In particular, according to the present invention, there can be provided a material which enables realization of high sensitivity by using a simple magnetic biosensor such as a semiconductor Hall element and a magnetoresistance effect element.

### BACKGROUND OF THE INVENTION

1. Field of the Invention

The present invention relates to a material for improving reaction efficiency and detection sensitivity of a sensor to detect the presence and concentration of target substances in a test solution and a method thereof.

2. Description of the Related Art

A biosensor is a measurement device exploiting molecular recognition ability possessed by living bodies or biomolecules. In a living body, examples of pairs of substances having affinity for each other include enzyme-substrate, antigen-antibody and DNA-DNA combinations. A biosensor exploits a principle that one substance of such combination can be selectively measured by using the other substance being immobilized or supported to a substrate.

In recent years, a biosensor is expected to be widely applied not only in the field of medicine but also in environment, food products and the like. In order to expand its area of use, there is a demand for a biosensor being small and light to be installed in any place and be portable, and having high sensitivity.

At present, there are many researches conducted on a biosensor to detect the presence and concentration of target substances in a test solution by using a magnetic sensor to detect the presence and number of a magnetic marker locating in the vicinity of the surface of the detection region as one high sensitive sensing method. The method is also used for solid phase analysis.

FIG. 1 illustrates an example of solid phase analysis method using a conventional magnetic marker. In the method illustrated in FIG. 1, first, a first capture agent composition is immobilized to a substrate surface in advance, wherein the first capture agent composition (which is referred to a primary antibody in the case of an antigen antibody reaction) can specifically recognize and capture one region of a target substance (which is referred to an epitope in the case of an antigen antibody reaction). Next, a test solution containing a target substance is come into contact with the substrate. Through this operation, the target substance is specifically captured by the first capture agent composition. Subsequently, a magnetic marker including a second capture agent composition (which is referred to a secondary antibody in the case of an antigen antibody reaction) is introduced in the solution, wherein the second capture agent composition can specifically recognize and capture the other region of the target substance specifically captured by the first capture agent composition. Through this operation, the second capture agent composition is specifically captured by the target substance which is specifically captured by the first capture agent composition being immobilized to the substrate surface (sandwich method). As a result, as illustrated in FIG. 1, the magnetic marker is immobilized to the substrate surface via the target substance.

In addition, as a different method, a magnetic marker including a second capture agent composition is added into the test solution containing the target substance in advance to form the “target substance-second capture agent composition” complex. The complex is come into contact with the first capture agent composition immobilized to the substrate. As a result, as illustrated in FIG. 1, the magnetic marker can also be immobilized to the substrate surface via the target substance.

Consequently, by measuring the number of such magnetic marker immobilized to the substrate surface by some kind of method, the number or concentration of the target substance of one's interest can be calculated.

The following method has been proposed as a biosensor using such magnetic detection method.

Japanese Patent Application Laid-Open No. 2001-033455 discloses an immunological test method in which a magnetic material used as a labeling agent is bound to a target substance contained in a test solution through an antigen antibody reaction, the labeling agent is then magnetized and detected by SQUID (superconducting quantum interference device) used as a magnetic sensor. The document discloses that the magnetic marker to be used herein is defined to be in the range of 40 to 100 nm in size by coating a magnetic particle in the range of 20 to 40 nm with a polymer so that it contributes to improve sensitivity of the aforementioned SQUID. Although the aforementioned SQUID has extremely high detection sensitivity, issues remain in terms of requiring a cryogenic environment by using liquid helium for conducting detection, difficulty in size and weight reduction, and requiring a high running cost.

Consequently, in order to solve the aforementioned problems, a biosensor using a magnetic sensor which is small and capable of measurements at room temperature has been proposed. There are many kinds of small magnetic sensors which can detect magnetic particles. Examples include a Hall effect element (WO2003/067258), a magnetoresistance effect element (U.S. Pat. No. 5,981,297) and a magnetic impedance element (Japanese Patent Application Laid-Open No. H10-234694).

The magnetic field H formed by a magnetic sphere at the point position P on the vector r from the center of the magnetic sphere can be described by the following formula.

$$H = \frac{1}{4 π μ {r}^{3}} \{ \frac{3}{{r}^{2}} ( mr ) r - m \} \qquad [ Formula 1 ]$$

As used herein, μ refers to magnetic permeability and m refers to magnetic moment by replacing magnetization of the magnetic sphere with a small magnet positioned at the center of the magnetic sphere. The formula indicates that, if the value of magnetization per unit volume is constant, the larger the volume of a magnetic sphere, the greater the value of m and the larger the magnetic field applied to the point P. Therefore, when a magnetic marker is detected by a magnetic sensor, the magnetic marker is preferably large in its particle diameter to favorably conduct detection. However, large particle diameter may deteriorate reactivity and dispersibility thereof, and interfere with the use as a biosensor.

### SUMMARY OF THE INVENTION

The present invention relates to a biosensor using a magnetic sensor, a method for improving detection sensitivity while maintaining reactivity and dispersibility thereof. In particular, the present invention provides a method for enabling realization of high sensitivity by using a simple magnetic biosensor such as a semiconductor Hall element and a magnetoresistance effect element.

The capture agent member of the present invention to solve the aforementioned problems is as follows.

The first aspect of the present invention relates to a capture agent member to be used for a magnetic sensor to detect the presence or concentration of a target substance in a test solution by detecting the presence or number of a magnetic marker, wherein the capture agent member includes a capture agent to capture the target substance and a labeling agent serving as a nucleus for the magnetic marker to agglutinate, and wherein the capture agent is labeled with the labeling agent.

The second aspect of the present invention relates to a method for detecting the presence or concentration of a target substance in a test solution by detecting the presence or number of the magnetic marker, wherein the detection method includes reacting a first capture agent which captures the target substance in the test solution with the target substance, reacting a capture agent member including a second capture agent which captures the target substance with the target substance being reacted with the first capture agent, and causing agglutination of a magnetic marker with a nucleus of a labeling agent used to at least partially label the capture agent member including the second capture agent.

The third aspect of the present invention relates to a kit to be used for a magnetic sensor to detect the presence or concentration of a target substance in a test solution by detecting the presence or number of the magnetic marker, wherein the kit includes a sensor element, a capture agent member and a magnetic marker, wherein the sensor element includes a sensor element member and a first capture agent which is immobilized to the surface of the member and captures the target substance, the capture agent member includes a second capture agent for capturing the target substance and the labeling agent having a surface charge in the test solution, and the magnetic marker includes a magnetic material having a charge whose polarity differs from that of the labeling agent or, alternatively, includes the magnetic material and a material having a charge whose polarity differs from that of the labeling agent.

The fourth aspect of the present invention relates to a kit to be used for a magnetic sensor to detect the presence or concentration of a target substance in a test solution by detecting the presence or number of the magnetic marker, wherein the kit includes a sensor element, a capture agent member and a magnetic marker, wherein the sensor element includes the sensor element member and a first capture agent which is immobilized to the surface of the member and captures the target substance, the capture agent member includes a second capture agent for capturing the target substance and the labeling agent at least partially containing a material specific peptide, and the magnetic marker includes the magnetic material and a material having affinity for the material specific peptide.

The fifth aspect of the present invention relates to a kit to be used for a magnetic sensor to detect the presence or concentration of a target substance in a test solution by detecting the presence or number of the magnetic marker, wherein the kit includes a sensor element, a capture agent member and a magnetic marker, wherein the sensor element includes the sensor element member and a first capture agent which is immobilized to the surface of the member and captures the target substance, the capture agent member includes a second capture agent for capturing the target substance and the labeling agent at least partially containing a stimuli responsive polymer, and the magnetic marker includes a magnetic material and the stimuli responsive polymer.

Further features of the present invention will become apparent from the following description of exemplary embodiments with reference to the attached drawings.

### BRIEF DESCRIPTION OF THE DRAWINGS

FIG. 1 is a view showing a frame format of an example of solid phase analysis method by using a conventional magnetic marker.

FIG. 2 is a view showing a frame format of an example of the element in the biosensor of the present invention.

FIGS. 3A, 3B, 3C, 3D, 3E, 3F and 3G are views showing a frame format of a process for manufacturing the magnetoresistance effect element to be used in an embodiment of the present invention.

FIG. 4 is a view showing a frame format of a detection circuit to be used in an embodiment of the present invention.

FIG. 5 is a view showing a frame format of a capture agent member.

FIG. 6 is a view showing a frame format of a detection circuit of a biosensor to be used in an embodiment of the present invention, wherein a Hall effect element is used as the sensor element.

FIG. 7 is a view showing a frame format illustrating the configuration of a biosensor to be used in an embodiment of the present invention, wherein a magnetic impedance element is used as the sensor element.

FIG. 8 is a view showing a frame format of a detection circuit of a biosensor to be used in an embodiment of the present invention, wherein a magnetic impedance element is used as the sensor element.

### DESCRIPTION OF THE EMBODIMENTS

Preferred embodiments of the present invention will now be described in detail in accordance with the accompanying drawings.

The present invention relates to a capture agent member to be used for a magnetic sensor to detect the presence or concentration of a target substance in a test solution by detecting the presence or number of a magnetic marker, wherein the capture agent member includes a labeling agent serving as a nucleus for the magnetic marker to agglutinate.

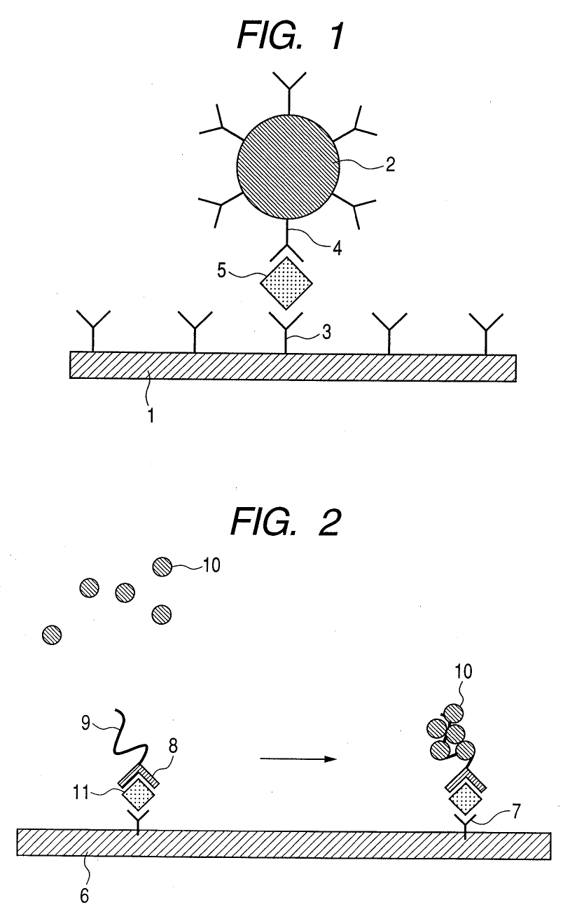

FIG. 1 illustrates a view showing a frame format of an example of a method for detecting a target substance with a sensor preferably using the capture agent member of the present invention. In FIG. 1, a first capture agent immobilized to the sensor element and a second capture agent member to be labeled with a labeling agent undergo a sandwich-type antigen antibody reaction via the target substance. Agglutination of a magnetic marker with a nucleus of a labeling agent immobilized to the second capture agent member binding to the target substance allows detection of the magnetic signals emitted from the magnetic marker immobilized to the sensor element. As a result, the present or concentration of the target substance in the test solution can be detected.

<<Capture Agent Member>>

In the present invention, a capture agent member includes at least the after-mentioned capture molecule and labeling agent.

<<Capture Molecule>>

In the present invention, a capture molecule refers to a substance involved in selection of the target substance in the test solution. Examples include a substance which directly and selectively reacts with the target substance in the test solution (namely, a receptor) and a substance involved in a reaction of the target substance (such as a substance exerting its catalytic function for a reaction of the target substance selectively). In addition, the capture molecule may also exert functions involved in display of the presence and degree of detection. For example, the capture molecule may exert a chromophore function by reacting with a substance released from the receptor and residual substances. The capture molecule to be used in the present invention includes, without limitation, enzymes, sugar chains, catalysts, antibodies, antibody fragments, antigens, nucleic acids and color agents.

Moreover, the detection target in the present invention does not necessarily have to be the target substance with which the capture molecule directly reacts, and may be a substance which can be measured indirectly. For example, the detection target can be measured by detecting the target substance present specifically in the detection target. Therefore, the detection target is not limited to a biomaterial, and the size thereof is not limited either. However, preferred target substances are biomaterials included in a living body such as sugars, proteins, amino acids, antibodies, antigens, pseudo antigens, vitamins and nucleic acids, related substances thereof and artificially synthesized pseudo biomaterials.

Furthermore, the capture agent composition can be used in combination. As a capture molecule in the present invention, capture molecules such as a complex enzyme and an antigen-enzyme can be composed.

<<Labeling Agent>>

In the present invention, a labeling agent refers to a material capable of selectively causing agglutination of the after-mentioned magnetic marker to the vicinity thereof.

Examples of preferred material which can be used for the labeling agent include inorganic materials having a surface charge in the test solution, organic polymers and amino acids. The isoelectric point of the material having a surface charge is preferably different from the pH of the test solution in terms of charge intensity. In addition, a peptide or a nucleic acid having affinity for the material partially included in the after-mentioned magnetic marker can also be used as the labeling agent. Moreover, a material exhibiting phase transition according to micro changes in the external environment (stimuli) can also be used. Example of such material includes a stimuli responsive polymer.

<<Material Having a Charge>>

<Inorganic Material>

Examples of inorganic material which can be used in the present invention include magnesium oxide (MgO: Isoelectric point 12.4), zinc oxide (ZnO: ditto 9.3), ferrite (α-Fe₂O₃: ditto 9.04) and silicon dioxide (SiO₂: ditto 1.8). Materials other than the inorganic oxides can be used without being limited to the aforementioned examples as long as their isoelectric points are different from the pH of the test solution and have a charge in the test solution.

<Organic Material>

Polymer including a carboxyl group, amino group or phosphoryl group on the side chain can be used as a labeling agent of the present invention. Examples of such pH sensitive material include polymers based on pH sensitive vinyl monomers such as acrylic acid, methacrylic acid, maleic acid, maleic acid anhydride, 2-acrylamide-2-methyl-1-propanesulfonic acid, aminoethyl methacrylate, phosphorylethyl acrylate or phosphorylethyl methacrylate. Copolymers consisting of polymers of such pH-sensitive vinyl monomers and 2 kinds or more of the monomers can also be used.

<Amino Acid>

In addition, the labeling agent can be an amino acid or a peptide. Examples of amino acid which can be preferably used as a labeling agent having a positive charge in the present invention include basic amino acids such as arginine (isoelectric point 10.76) and lysine (isoelectric point 9.74). On the other hand, examples of labeling agent having a negative charge include acidic amino acids such as asparagine acid (isoelectric point 2.77) and glutamic acid (isoelectric point 3.22).

<<Material Having Affinity>>

<Material Specific Peptide>

Material specific peptide can be used as a labeling agent. Example of material specific peptide includes a ferrite specific peptide. It is known that the surface presentation method in which a material specific peptide is presented on a coat protein of a virus as represented by M13 phase and the surface of cells such as E. coli and yeast can be used as a method for selecting such material specific peptide. In addition, there are enzymes which generate enzyme-suicidal substrate complexes by deactivating its original enzyme catalytic functions owning to an interaction with a suicidal substrate. For example, a suicidal substrate protein Barstar is known to tightly bind with the RNA degrading enzyme Barnase to inhibit its activity. An enzyme or a suicidal substrate forming such enzyme-suicidal substrate complex can be used as a labeling agent of the present invention. When an enzyme is used as a labeling agent, a magnetic marker can bind with the labeling agent by modifying the surface of the magnetic marker with a suicidal substrate of the enzyme. Therefore, agglutination of the magnetic marker to the capture agent can occur by labeling the capture agent with a plurality of enzymes. Moreover, a protein including a plurality of binding domains for a specific substance within the molecule (polyvalent protein) can also be used as a labeling agent in the present invention. When such polyvalent protein is used as a labeling agent in the present invention, improvement in quantitative performance can be expected. For example, when a material specific peptide such as the ferrite specific peptide is used as a labeling agent, it is difficult to regulate the number of the magnetic marker to which the peptide binds. Therefore, there is a possibility that the number of the capture agent member which captures the target on the sensor wafer does not correlate with the number of the magnetic marker actually detected. However, when (strept) avidin, which is a polyvalent protein, is used as a labeling agent, one (strept) avidin molecule can bind with a maximum of 4 biotin molecules because it has 4 subunits capable of binding with one biotin molecule. However, steric barrier may be induced depending on the particle diameter of the magnetic marker. Thus, 1 to 4 magnetic markers can bind per (strept) avidin molecule. Therefore, the number of the magnetic marker which agglutinates to the capture agent member can be regulated and improvement in quantitative performance can be expected by giving consideration to the particle diameter of the magnetic marker to be used. In addition, the distance from the surface of the magnetic marker to biotin used to modify the surface of the magnetic marker may affect quantitative performance. Biotin used to modify the magnetic marker can modify it via a linker. However, when the distance from the surface of the magnetic marker to biotin is too far, a plurality of biotin binding sites of a (strept) avidin molecule to be used as a labeling agent for one molecule of the capture agent member bound to the target substance on the sensor wafer may be bound by biotin on the same magnetic marker. Therefore, the length of a linker is preferably regulated so that a plurality of biotins being present on the identical magnetic marker does not bind to one (strept) avidin molecule in terms of improving quantitative performance. Moreover, quantitative performance can also be improved by regulating the density of biotin modification on the surface of the magnetic marker. For example, when one magnetic marker is modified with one biotin molecule, it is preferred because biotin being present on the identical magnetic marker cannot bind to a plurality of biotin binding subunits in one (strept) avidin molecule. Furthermore, it is known that (strept) avidin is in the range of 6 nm in size. When a plurality of biotins is present on the magnetic marker, it is preferred because one biotin binding subunit of (strept) avidin and the magnetic market can bind on a one to one basis when the distance between biotins on the identical magnetic marker is regulated so that biotins are present at a distance that cannot bind to a plurality of subunits on avidin.

<Nucleic Acid>

A single strand DNA and/or single strand RNA can also be used as a labeling agent in the present invention. A single strand DNA and/or single strand RNA can anneal to a single strand DNA and/or single strand RNA having a sequence complementary to the single strand DNA and/or single strand RNA at a temperature in the range of the Tm value or lower. Therefore, such single strand DNA and/or single strand RNA can be used as a labeling agent in the present invention. The single strand DNA and/or single strand RNA to be used as a labeling agent complementarily anneals to a single strand DNA and/or single strand RNA immobilized to the after-mentioned magnetic marker. The sequence and length thereof are not specifically limited as long as non-specific annealing does not occur with sequences other than the complementary single strand DNA and/or single strand RNA. As for the method of preparing such single strand DNA and/or single strand RNA, a specific single strand DNA can be prepared by using Exonuclease III and Lambda exonuclease. Alternatively, one of DNA nuclease and RNA nuclease can also be used to digest one of the DNA and RNA after preparation of a DNA/RNA hybrid to prepare one of single strand DNA and single strand RNA.

<<Stimuli Responsive Material>>

<Stimuli Responsive Polymer>

In the present invention, a stimuli responsive polymer refers to a polymer exhibiting phase transition according to stimuli.

<Stimulus>

In the present invention, a stimulus refers to a micro change in the external environment (the environment in which the test solution of the present invention is present) and, more specifically, includes changes of temperature and pH and light irradiation.

<<Material of a Stimuli Responsive Polymer>>

As a stimuli responsive polymer to be used as a labeling agent in the present invention, the following can be used. Examples of temperature responsive polymer include a polymer having an upper critical solution temperature and a polymer having a lower critical solution temperature. Examples of polymer having an upper critical solution temperature include polymers consisting of at least one monomer selected from the group consisting of acrylamide, acetyl acrylamide, biotinol acrylate, N-biotinyl-N′-methacroyl trimethylene amide, acroyl glycine amide, acroyl sarcosine amide, methacryl sarcosine amide, acroyl nipecotamide and acroyl methyl uracil. In addition, copolymers consisting of at least 2 kinds of these monomers can also be used.

On the other hand, examples of polymer having a lower critical solution temperature include polymers consisting of N-substituted (metha)acrylamide derivatives such as N-n-propylacrylamide, N-isopropylacrylamide, N-ethylacrylamide, N,N-dimethylacrylamide, N-acryloyl pyrrolidine, N-acryloyl piperidine, N-acryloyl morpholine, N-n-propyl methacryl amide, N-isopropyl methacryl amide, N-ethyl methacryl amide, N,N-dimethyl methacryl amide, N-methacryloyl pyrrolidine, N-methacryloyl piperidine and N-methacryloyl morpholine; polyoxyethylene alkylamine derivatives such as hydroxypropyl cellulose, polyvinyl alcohol partial acetification products, polyvinyl methylether, (polyoxyethylene-polyoxypropylene) block copolymer and polyoxyethylene lauryl amine; polyoxyethylene sorbitan ester derivatives such as polyoxyethylene sorbitan laurate; (polyoxyethylene alkylphenylether)(metha)acrylates such as (polyoxyethylene nonylphenyl ether)acrylate and (polyoxyethylene octylphenyl ether)methacrylate; and polyoxyethylene(metha)acrylic acid ester derivatives such as (polyoxyethylene alkylether)(metha)acrylates such as (polyoxyethylene laurylether)acrylate and (polyoxyethylene oleylether)methacrylate. In addition, copolymers consisting of polymers thereof and at least 2 kinds of these monomers can also be used.

A light sensitive polymer generally includes a chromogenic group on the side chain of the polymer. Typical example of chromogenic group includes an aromatic diazo dye (Ciardelli, Biopolymers 23, 1423-1437 (1984); Kungwatchakun et al., Makromol. Chem., Rapid Commun. 9, 243-246 (1988); Lohmann et al., CRC Crit. Rev. Therap. Drug Carrier Systems 5, 263 (1989); Mamada et al., and Macromolecules 23, 1517 (1990)). When the aromatic diazo dye is exposed to UV light in the range of 350 to 410 nm, polymer conformation change is induced by being isomerized from the comparably hydrophobic trans form to the bipolar, more hydrophilic cis form. Owning to this change, a polymer solution which has been cloudy becomes transparent according to the degree of chromocomplex to the skeleton and the water solubility of the main unit of the skeleton. On the other hand, the reverse phenomenon can be induced by exposing it to a visible light of approximately 750 nm.

A pH responsive polymer generally includes a pH sensitive group on the side chain such as a —OPO(OH)₂ group, —COOH group or —NH₂ group. Typical examples of pH sensitive material include polymers based on pH sensitive vinyl monomers such as acrylic acid, methacrylic acid, maleic acid, maleic acid anhydride, 2-acrylamide-2-metyl-1-propanesulfonic acid, aminoethyl methacrylate, phosphorylethyl acrylate or phosphorylethyl methacrylate. Polymers including the pH sensitive groups can cause phase separation owning to changes of pH in the test solution.

<<Method for Immobilizing the Labeling Agent to the Capture Molecule>>

Various methods can be used as a method for immobilizing the labeling agent to the capture molecule, depending on material of the labeling agent. The location at which the labeling agent is immobilized to the capture molecule and the method of immobilization are not specifically limited as long as the method does not inhibit the binding ability of the capture molecule to the captured molecule and the labeling agent can serve as a nucleus to cause agglutination of the after-mentioned magnetic marker. For example, when the capture molecule is a protein, the labeling agent can be immobilized to the capture molecule at the carboxyl terminus, amino terminus and/or a random location as long as it does not inhibit the functions of the capture molecule. In addition, as an example of method for immobilizing the labeling agent to the capture molecule, a method of producing it by physical adsorption and/or chemical bonding can be cited and a method of producing it as a fusion peptide by gene linking can be cited.

<<Physical Adsorption>>

Physical adsorption of the labeling agent to the capture molecule enables non-specific absorption by mixing the capture molecule and labeling agent. It is preferred in terms of ease of operation.

<<Chemical Bonding>>

On the other hand, as a method for immobilizing the labeling agent to the capture molecule, chemical bonding such as covalent bonding can be applied. The chemical bonding is preferred because the bonding is strong in comparison with that of physical absorption. For example, when the capture molecule is a protein, the amino group of the amino acid contained in the protein sequence can be immobilized to the carboxyl group immobilized to the material surface of the labeling agent by a conventionally known method in the field of the present invention as a method for covalently immobilizing the labeling agent to the capture molecule.

<<Fusion Peptide by Gene Linking>>

In addition, when the capture molecule and labeling agent are proteins, peptides and amino acids, the labeling agent can be immobilized to the capture molecule as a fusion polypeptide of the capture molecule and labeling agent at the carboxyl terminus and/or amino terminus of the capture molecule or at a random location. A conventionally known method for chemical synthesis or synthesis using a living body in the technical field of the present invention can be uses as a method for preparing the fusion polypeptide.

<<Composition of the Labeling Agent>>

<When a Metal is Used as the Labeling Agent>

When a metal is used as the labeling agent to be used in the present invention, the average particle diameter is preferably 1 nm or more and less than 1000 nm without specific limitation. The average particle diameter is particularly preferably 3 nm or more and less than 200 nm in order to improve recognition performance and reactivity of the capture agent with labeling agent. Metallic material to be used is not limited as long as the material has a charge in the test solution. Examples of such metallic material include magnesium oxide (MgO: Isoelectric point 12.4), zinc oxide (ZnO: ditto 9.3), ferrite (α-Fe₂O₃: ditto 9.04) and silicon dioxide (SiO₂: ditto 1.8). In addition, the metallic material can be partially or entirely coated with a hydrophilic polymer in order to obtain favorable dispersibility in the test solution as long as the coating does not impair the polarity.

<When a Polymer is Used as a Labeling Agent>

A charged polymer can also be used as a labeling agent in the present invention. As a polymer having a charge, polymers including an amino group, carboxy group and phosphoryl group on the side chain can be used. Examples of such polymer include polymers based on pH-sensitive vinyl monomers such as acrylic acid, methacrylic acid, maleic acid, maleic acid anhydride, 2-acrylamide-2-methyl-1-propanesulfonic acid, aminoethyl methacrylate, phosphorylethyl acrylate or phosphorylethyl methacrylate. Copolymers consisting of polymers of such pH-sensitive vinyl monomers and 2 kinds or more of the monomers can also be used. Such polymer can be covalently immobilized to a portion of the capture agent. For example, when an antibody is used as the capture agent, immobilization can be conducted by reacting the amino group contained in the antibody and the carboxy group contained in the polymer side chain by a conventionally known method in the field of the present invention. Moreover, a polymer coated metallic material obtained by coating the metallic oxide with the polymer having polarity can be used as a labeling agent in the present invention. Furthermore, a metallic material obtained by coating the metallic material with a hydrophilic polymer, thereby improving dispersibility, and then introducing an amino group or carboxy group to the terminus of the hydrophilic polymer can be used as a labeling agent in the present invention.

<When an Amino Acid is Used as a Labeling Agent>

When the capture molecule and labeling agent are proteins or peptides, and the labeling agent is an amino acid, the labeling agent can be immobilized to the capture molecule as a fusion polypeptide of the capture molecule and labeling agent at the carboxy terminus and/or amino terminus of the capture molecule or at a random location. Although an amino acid can be used as the labeling agent singularly, it can be used as a polyamino acid linking a plurality of the identical or different amino acids, wherein the polyamino acid has the identical polarity in the test solution. In such cases, the polyamino acid is preferred because it can expand the region having a charge and facilitate agglutination of the after-mentioned magnetic marker to the vicinity of the labeling agent in comparison with the case where one amino acid is being used as the labeling agent. In addition, a polyamino acid to be used as the labeling agent can also be linked by a linker. The linker can have any sequence as long as it does not inhibit agglutination of the magnetic marker with a nucleus of the labeling agent. For example, it is preferred to use a material having weak polarity in the test solution as the linker because it electrostatically attracts the magnetic marker contained in the labeling agent and facilitates agglutination of the magnetic marker to the vicinity of the amino acid which serves as a region for agglutination. As an example of such linker, a conventional linker such as SEQ ID NO:5 can be used. Moreover, regarding the structure of an amino acid to be used as the labeling agent, a dendrimer structure can be used. A dendrimer is consisting of the central molecule termed “core” and a side chain region termed “dendron.” The number of branching in the dendron region is termed “generation.” Dendritic polylysine is known as an example of dendrimer using an amino acid for a dendron in the dendrimer structure, and can be used as the labeling agent in the present invention.

<When a Material Specific Peptide is Used as a Labeling Agent>

When the capture molecule and labeling agent are proteins or peptides, and the labeling agent is an amino acid, the labeling agent can be immobilized to the capture molecule as a fusion polypeptide of the capture molecule and labeling agent at the carboxy terminus and/or amino terminus of the capture molecule or at a random location. Although a material specific peptide can be used as the labeling agent singularly, it can be repeatedly linked with or without a linker to be used. It is preferred to repeatedly link a material specific peptide because it enables a tighter agglutination of the magnetic marker owning to expansion of the region to which the magnetic marker can bind and improvement in binding ability. In addition, a more quantitative agglutination can be performed by regulating the number of the material specific peptide to be repeatedly linked as it can regulate the number of the agglutinating magnetic marker which agglutinates via the material specific peptide. Moreover, a material specific peptide which only binds to one material contained in the magnetic marker can be used singularly or repeatedly as the labeling agent. Furthermore, material specific peptides having affinity for 2 materials or more contained in the magnetic marker can also be fused to be used.

<<Magnetic Marker>>

Regarding the magnetic marker to be used in the present invention, the size of a marker can be diversely selected according to the configuration and size of a sensor element and the purpose of use and is not specifically limited. The size is preferably in the range of a few nm to a few hundreds nm, and more preferably in the range from 10 nm to 200 nm in order to favorably maintain dispersibility. In addition, the average particle diameter of magnetic microparticles can be measured by dynamic light scattering method.

Examples of magnetic microparticle constituting such magnetic marker include microparticles such as ferrite, nickel oxide, ferrocobalt oxide, barium ferrite, carbon steel, tungsten steel, KS steel, rare earth-cobalt magnet and hematite. In particular, ferrite is preferred because it has sufficient magnetism under bioactive conditions and is resistant to deterioration such as oxidation in a solvent. Ferrite is selected from a group consisting of ferrioxides such as magnetite (Fe₃O₄), hematite (α-Fe₂O₃) and maghemite (γ-Fe₂O₃) and complexes thereof in which a portion of Fe is substituted with other atoms. As other atoms, at least one atom selected from the group consisting of Li, Mg, Al, Si, Ca, Sc, Ti, V, Cr, Mn, Co, Ni, Cu, Zn, Ga, Ge, Zr, Nb, Mo, Cd, In, Sn, Ta and W can be cited.

<Composition When Agglutination is Caused by Electrostatic Interaction>

As an example of composition of the magnetic marker to be used when the labeling agent of the capture agent member has a surface charge in the test solution, a magnetic marker at least partially containing ferrite is preferably used. Because the isoelectric point of hematite (α-Fe₂O₃) is 9.04, it can cause agglutination of ferrite particles with a nucleus of the labeling agent as long as the ferrite particles do not form self-agglutination in the test solution when using the capture agent member to be labeled with a material having a surface charge different from the surface charge of ferrite in the test solution. In such cases, performing other treatments on the surface of the ferrite particles is not required. Therefore, it has an advantage of being able to simplify particle preparation.

A polar group such as an amino group and carboxy group can also be immobilized to the surface of the magnetic microparticle. It is preferred to immobilize a polar group on the surface of the magnetic microparticle because the surface of the magnetic marker then can have polarity even when the pH suitable for the capture agent member to capture the target substance is the isoelectric point of the magnetic microparticle. A conventionally known method in the technical field of the present invention can be uses as a method for immobilizing an amino group or a carboxy group to the surface of the magnetic microparticle. In addition, the surface of the magnetic microparticle can be coated with a hydrophilic polymer such as polyethyleneglycol to immobilize a polar group such as an amino group or a carboxy group to the terminus of the hydrophilic polymer. It is preferred to coat the magnetic microparticle with the hydrophilic polymer because it can improve dispersibility of the magnetic market in the test solution.

<Composition When Agglutination is Caused by Molecular Recognition>

As for the magnetic marker to be used when the labeling agent of the capture agent member is a material specific peptide, the surface of the magnetic marker can at least partially contain a material recognized by the material specific peptide. Regarding examples of such material, the ferrite and alloy of ferrite and other metallic material such as barium ferrite can be used as the magnetic microparticle when a ferrite specific peptide is used as the labeling agent. In addition, the magnetic marker can also be coated with a phydrophilic polymer and the like in order to improve dispersibility as long as it does not impair affinity for the material specific peptide. Moreover, regarding the composition of the magnetic marker when an enzyme or (strept) avidin is used as an specific peptide, the surface of the magnetic marker can be at least partially modified with a suicidal substrate for the enzyme, biotin or StreptoTag. The magnetic marker can also be modified with the suicidal substrate, biotin or StreptoTag via a linker. Furthermore, a conventionally known method in the field of the present invention such as physical absorption and chemical crosslink can be used as a modification method. Any method can be used as long as it has binding ability with the enzyme or (strept) avidin to be used as a specific peptide.

<Composition When Agglutination is Caused by a Stimuli Responsive Polymer>

As for the magnetic marker to be used when the labeling agent of the capture agent member is a stimuli responsive polymer, a magnetic marker at least partially containing a stimuli responsive polymer can be used. As such stimuli responsive polymer which can be used as the magnetic marker, a temperature responsive polymer, light responsive polymer and pH responsive polymer can be used. A method for coating and immobilizing a stimuli responsive polymer to the magnetic microparticle can be conducted by a conventionally known method in the technical field of the present invention. For example, Japanese Patent Application Laid-Open No. 2005-082538 discloses a method for immobilizing a heat responsive polymer to a magnetic particle via a polyvalent alcohol and polyvalent alcohol derivative.

Examples of temperature responsive polymer include a polymer having an upper critical solution temperature and a polymer having a lower critical solution temperature. Examples of polymer having an upper critical solution temperature include polymers consisting of at least one monomer selected from the group consisting of acrylamide, acetyl acrylamide, biotinol acrylate, N-biotinyl-N′-methacroyl trimethylene amide, acroyl glycine amide, acroyl sarcosine amide, methacryl sarcosine amide, acroyl nipecotamide and acroyl methyl uracil. In addition, copolymers consisting of at least 2 kinds of these monomers can also be used.

On the other hand, examples of polymer having a lower critical solution temperature include polymers consisting of N-substituted (metha) acrylamide derivatives such as N-n-propylacrylamide, N-isopropylacrylamide, N-ethylacrylamide, N,N-dimethylacrylamide, N-acryloyl pyrrolidine, N-acryloyl piperidine, N-acryloyl morpholine, N-n-propyl methacryl amide, N-isopropyl methacryl amide, N-ethyl methacryl amide, N,N-dimethyl methacryl amide, N-methacryloyl pyrrolidine, N-methacryloyl piperidine and N-methacryloyl morpholine; polyoxyethylene alkylamine derivatives such as hydroxypropyl cellulose, polyvinyl alcohol partial acetification products, polyvinyl methylether, (polyoxyethylene-polyoxypropylene) block copolymer and polyoxyethylene lauryl amine; polyoxyethylene sorbitan ester derivatives such as polyoxyethylene sorbitan laurate; (polyoxyethylene alkylphenylether)(metha)acrylates such as (polyoxyethylene nonylphenyl ether)acrylate and (polyoxyethylene octylphenyl ether)methacrylate; and polyoxyethylene(metha)acrylic acid ester derivatives such as (polyoxyethylene alkylether)(metha)acrylates such as (polyoxyethylene laurylether)acrylate and (polyoxyethylene oleylether)methacrylate. In addition, copolymers consisting of polymers thereof and at least 2 kinds of these monomers can also be used.

A light sensitive polymer generally includes a chromogenic group on the side chain of the polymer. Typical example of chromogenic group includes an aromatic diazo dye (Ciardelli, Biopolymers 23, 1423-1437 (1984); Kungwatchakun et al., Makromol. Chem., Rapid Commun. 9, 243-246 (1988); Lohmann et al., CRC Crit. Rev. Therap. Drug Carrier Systems 5, 263 (1989); Mamada et al., and Macromolecules 23, 1517 (1990)). When the aromatic diazo dye is exposed to UV light in the range of 350 to 410 nm, polymer conformation change is induced by being isomerized from the comparably hydrophobic trans form to the bipolar, more hydrophilic cis form. Owning to this change, a polymer solution which has been cloudy becomes transparent according to the degree of chromocomplex to the skeleton and the water solubility of the main unit of the skeleton. On the other hand, the reverse phenomenon can be induced by exposing it to a visible light of approximately 750 nm.

A pH responsive polymer generally includes a pH sensitive group on the side chain such as a —OPO(OH)₂ group, —COOH group or —NH₂ group. Typical examples of pH sensitive material include polymers based on pH sensitive vinyl monomers such as acrylic acid, methacrylic acid, maleic acid, maleic acid anhydride, 2-acrylamide-2-metyl-1-propanesulfonic acid, aminoethyl methacrylate, phosphorylethyl acrylate or phosphorylethyl methacrylate. Polymers containing the pH sensitive groups can cause phase separation owning to changes of pH in the test solution.

The various stimuli responsive polymers can be used in any combination with the labeling agent immobilized to the capture agent member as long as it can agglutinate with a nucleus of the labeling agent by an external stimulus. In addition, the various stimuli responsive polymers can be coated with a hydrophilic polymer and the like for the purpose of improving dispersibility of the magnetic marker as long as the coating does not impair responsiveness to external stimuli.

<<A Method for Causing Agglutination of the Labeling Agent and Magnetic Marker>>

A method for causing agglutination of the labeling agent and magnetic marker in the present invention can be dissected separately in the following cases: when using a material having a surface charge in the test solution, when using a molecular recognition material, and when using a material agglutinating according to external stimuli.

<When a Material Having a Surface Charge in the Test Solution is Used>

When a material having a surface charge in the test solution is used as the labeling agent immobilized to the capture agent member, a magnetic marker having a surface charge different from that of the labeling agent in the test solution can be used as the magnetic marker. The magnetic marker can agglutinate with a nucleus of the labeling agent of the capture agent member by letting the capture agent member and the magnetic marker coexist. For example, when an antibody fragment having affinity for the target substance is used as the capture agent and polyaspartic acid is used as the labeling agent, a polypeptide is prepared by fusing a plurality of aspartic acids as the labeling agent to the amino terminus and/or carboxy terminus of the antibody fragment. Because the isoelectric point of aspartic acid is 2.77, it charges negatively in a pH 7.0 buffer, for example. Therefore, polyaspartic acid labeled to the carboxy terminus of the capture agent member exhibits a negative charge. When ferrite microparticles are added to a pH 7.0 buffer containing the capture agent member, because the isoelectric point of ferrite is 9.04, it charges positively in a pH 7.0 buffer. As a result, agglutination of the ferrite microparticles occurs with a nucleus of aspartic acid which is used as the labeling agent of the capture agent member, and agglutination of the magnetic marker with a nucleus of the labeling agent can be confirmed. In addition, polyaspartic acid to be used as the labeling agent can be repeatedly fused via a linker. It is preferred to repeatedly fuse the polyaspartic acid because it can expand the region to which the positively charged magnetic marker can agglutinate by enabling a plurality of negatively charged regions to exist via a linker.

<When a Molecular Recognition Material is Used>

When a material specific peptide is used as the labeling agent immobilized to the capture agent member, a magnetic marker whose surface at least partially contains a material which is recognized by the material specific peptide can be used as the magnetic marker. The magnetic marker can agglutinate with a nucleus of the labeling agent to be immobilized to the capture agent member by letting the capture agent member to be labeled with the material specific peptide and the magnetic marker coexist.

For example, when an antibody fragment having affinity for the target substance is used as the capture agent, a polypeptide is prepared by fusing a ferrite specific peptide to the carboxy terminus of the antibody fragment via a linker. By adding ferrite microparticles to a solution of the ferrite specific peptide fusion polypeptide, agglutination of the ferrite particles occurs to the ferrite specific peptide, and agglutination of the magnetic marker to the labeling site of the capture agent member can be confirmed. In addition, a plurality of ferrite specific peptide units can be immobilized to the capture agent member via a linker. Affinity for ferrite is expected to be improved by repeating a plurality of ferrite specific peptide units in this way. Moreover, it is also expected to regulate the number of the magnetic marker to agglutinate by arbitrarily changing the number of repeats of the ferrite specific peptide and the total number of the ferrite specific peptide contained the labeling agent.

In addition, for example, a complex of a metallic nanoparticle and a material specific peptide can be used as the labeling agent. For example, a gold nanoparticle containing the ferrite specific peptide immobilized to the surface thereof by physical absorption can be used as the labeling agent. By fusing a cysteine residue to the amino terminus or carboxy terminus of a ferrite specific peptide, the ferrite specific peptide can be immobilized to the gold nanoparticle via a thiol group of the cysteine. As described above, the gold nanoparticle containing the ferrite specific peptide immobilized to the surface thereof can be physically absorbed and immobilized to an antibody having affinity for the target substance to prepare a capture agent member. When such labeling agent is used, for example, the size of the gold nanoparticle is selected in order to expect to select the number of the ferrite specific peptide immobilized to the gold nanoparticle and to expect to regulate the number of the agglutinating magnetic marker.

<When a Material Agglutinating According to External Stimuli>

When a stimuli responsive polymer is used as the labeling agent to be immobilized to the capture agent member, a magnetic marker at least partially containing the stimuli responsive polymer and a stimuli responsive polymer agglutinating according to external stimuli on the surface of the magnetic marker can be used as the magnetic marker. The magnetic marker can agglutinate to the labeling agent of the capture agent member by letting the capture agent member and the magnetic marker coexist and by adding an external stimulus suited to the stimuli responsive polymer.

For example, by using an antibody having affinity for the target substance as the capture agent, the magnetic particle coated with the stimuli responsive polymer serving as the labeling agent is immobilized by physical absorption to at least a portion of the antibody. In addition, a monomer including a functional group such as carboxylic acid, an amino group or an epoxy group is copolymerized with other monomers at the time of polymerization of the stimuli responsive polymer. A method of immobilizing the antibody to the polymer via the functional group can then be applied according to a conventionally known method in the technical field of the present invention. By these methods, the antibody labeled by the stimuli responsive polymer as the labeling agent is obtained and can be used as the capture agent member in the present invention. For example, when the stimuli responsive polymer is a polymer having a lower critical solution temperature, the capture agent member and the magnetic microparticle coated with the polymer having a lower critical solution temperature used as the magnetic marker are mixed at a temperature lower than or equal to the lower critical solution temperature. Under this condition, the capture agent member and the magnetic marker are maintained in a favorable dispersion state. The mixed solution is heated above the lower critical temperature to allow gelatification of the polymer having a lower critical solution temperature. As a result, agglutination of the magnetic marker and the labeling agent of the capture agent member can be confirmed. Alternatively, as an example of a light sensitive polymer being used as the stimuli responsive polymer, when the light sensitive polymer is an aromatic diazo dye, the capture agent member and the magnetic microparticle coated with the aromatic diazo dye used as the magnetic marker are mixed under exposure to UV light in the range of 350 to 410 nm. Because the aromatic diazo dye is known to exist in the more hydrophilic cis form under exposure to the UV light, the capture agent member and the magnetic marker are maintained in a favorable dispersion state in the reaction solution. By exposing the resulting mixed solution to a visible light of approximately 750 nm, for example, polymer conformation change in the aromatic diazo dye is induced by being isomerized to the comparably hydrophobic trans form. As a result, agglutination of the polymer solution is induced according to the degree of chromocomplex to the skeleton and the water solubility of the main unit of the skeleton, and agglutination of the capture agent member and the magnetic marker can be confirmed. When such light sensitive polymer is used as the labeling agent, the capture agent member may be denatured due to UV light exposure for a long time. Therefore, the mixing and agglutination processes are preferably completed within a timeframe which does not cause denaturation of the capture agent member. In addition, when a pH responsive polymer is used as the stimuli responsive polymer, the capture agent member and magnetic marker can agglutinate by the same method by introducing a pH change as an external stimulus. For example, the capture agent member and the magnetic microparticle coated with the pH responsive polymer are mixed in the pH range at which the pH responsive polymer maintains its water solubility and is dispersed in the solution. Subsequently, by changing the pH so that the pH responsive polymer exhibits phase change, agglutination of the labeling agent of the capture agent member and the magnetic marker can be induced. Various agglutination methods of the magnetic marker as described above can be used singularly or in combination. In addition, for example, a surfactant can be added to the reaction solution to suppress self-agglutination of the magnetic marker.

<<Detection Method by the Capture Agent Member>>

In a method for detecting the target substance in the test solution by using the capture agent member and magnetic marker in the present invention, the following are sequentially conducted: 1) reacting a first capture agent which captures the target substance in the test solution with the target substance; 2) reacting a capture agent member including a second capture agent which captures the target substance (which is referred to as “a member containing the second capture agent” hereinafter) with the target substance being reacted with the first capture agent; and 3) causing agglutination of a magnetic marker with a nucleus of a labeling agent used to at least partially label the second capture agent composition. The method can detect the presence of the target substance in the test solution by detecting the presence of the magnetic marker agglutinated to the capture agent member immobilized to the sensor element. A washing operation can be conducted between 1), 2) and 3) above. By conducting a washing operation, the signal-to-noise ratio is expected to be improved by removing the unreacted target substance and capture agent member. In addition, the operations in 1) and 2) can be conducted simultaneously. In such cases, it is preferable that the reaction time is expected to be reduced because 1) and 2) are conducted simultaneously. Moreover, a member containing the second capture agent and the target substance can be reacted with each other in advance to allow the complex of the member containing the second capture agent and the target substance to react with the first capture agent. As described above, regarding 1) and 2), the order of the operations is not specifically limited as long as a sandwich-like complex is formed by the first capture agent, the target substance and the member containing the second capture agent. On the other hand, by conducting 3) after formation of the sandwich-like complex, reactivity and dispersibility of the member containing the second capture agent and the target substance can be favorably maintained. 3) contributes to improve detection sensitivity by causing agglutination of the magnetic marker to a size detectable with a magnetic sensor.

As a method for detecting the target substance in the above processes, for example, a primary antibody is immobilized to a sensor element as a first capture agent. Subsequently, the test solution is drained dropwise to the detection region. By doing this, if the desired antigen is present in the test solution, the primary antibody and the antigen specifically bind to each other. Next, a secondary antibody labeled with the labeling agent is added to react with the target substance reacted with the primary antibody, and the secondary antibody is immobilized to the sensor element. Subsequently, by adding a magnetic marker agglutinating with a nucleus of the labeling agent immobilized to the secondary antibody, the magnetic marker agglutinates with a nucleus of the labeling agent immobilized to the capture agent member. Particles in the range of a few nm to a few hundreds nm in size are used as the magnetic marker in order to obtain favorable reactivity and dispersibility. In the case of a magnetic marker in such size, magnetism of the magnetic marker is known to be difficult to be detected with a simple magnetic sensor such as a semiconductor Hall element due to the influence of a heat noise. Therefore, the capture agent member and the magnetic marker of the present invention are combined in order to allow the magnetic marker to agglutinate with a nucleus of the labeling agent to a size detectable with a simple magnetic sensor such as a semiconductor Hall element, wherein the labeling agent is used to label the capture agent member immobilized by reacting with the target substance to the sensor. This enables for a magnetic sensor to indirectly detect the presence of the target molecule by detecting the magnetic marker. In addition, a washing operation can be conducted between the processes. It is preferable to include washing because it can remove impurities and the unreacted secondary antibody and the like contained in the test solution from the reaction system and, therefore, is effective in enhancing the signal-to-noise ratio.

<<Detection System>>

As a detection system using the biosensor of the present invention, any method can be used as long as the method detects the presence and concentration of the target substance in the test solution by detecting the presence and number of the magnetic marker located in the vicinity of the surface of the biosensor element. In particular, a system using magnetic effect is preferred. More specifically a magnetoresistance effect element, a Hall effect element, a magnetic impedance element and a superconducting quantum interference device element are preferably used.

<<Kit>>

A kit including the sensor element, the capture agent member and the magnetic marker can be used to enable the magnetic marker to agglutinate with a nucleus of the labeling agent contained in the capture agent member immobilized to the sensor element by capturing the target substance. By using the kit, the magnetic marker agglutinating to the sensor element can be detected by using a magnetic sensor such as a magnetoresistance effect element, a Hall effect element, a magnetic impedance element and a superconducting quantum interference device element in order to indirectly measure the presence and concentration of the target substance contained in the test solution.

The kit can be categorized into a kit used for the case where agglutination of the capture agent member and magnetic marker is conducted by an electrostatic action, a kit used for the case where agglutination of the capture agent member and magnetic marker is conducted by a molecular recognition material, and a kit used for the case where agglutination of the capture agent member and magnetic marker is conducted by an external stimulus as follows: a kit including a sensor element, a capture agent member and a magnetic marker, wherein the sensor element contains a first capture agent for capturing the target substance, the capture agent member contains the labeling agent having a surface charge in the test solution, and the magnetic marker has a charge whose polarity differs from that of the labeling agent; a kit including a sensor element, a capture agent member and a magnetic marker, wherein the sensor element contains a first capture agent for capturing the target substance, the capture agent member contains the labeling agent at least partially containing a material specific peptide, and the magnetic marker at least partially contains a material having affinity for the material specific peptide; and a kit including a sensor element, a capture agent member and a magnetic marker, wherein the sensor element contains a first capture agent for capturing the target substance, the capture agent member contains the labeling agent at least partially containing a stimuli responsive polymer, and the magnetic marker at least partially contains the stimuli responsive polymer.

### EXAMPLES

### Example 1

The present embodiment is an example to manufacture a magnetic marker at least partially containing the sensor element including a primary antibody capturing hen egg white lysozyme (HEL), a secondary antibody fragment capturing HEL labeled with polyaspartic acid, and ferrite particles, and to detect HEL as a sensor. In addition, a magnetoresistance effect element is used as the detection system of the sensor.

<<Detection Using Agglutination of a Magnetic Marker in Which a Material Having a Charge in the Test Solution is Used>>

(1) Preparation of an Anti-HEL Antibody Fragment Labeled With Polyaspartic Acid (Single Chain Fv: scfv)

(1-1) Production Process of a Fusion Polypeptide

A gene expression vector is constructed based on the pGEX-6P-1 (Amersham Biosciences Plc) vector to allow expression of the gene encoding the fusion polypeptide, wherein a scFv peptide of HyHEL10 represented by SEQ ID NO:1 (whose gene sequence is represented by SEQ ID NO:2) is bound to a polyaspartic acid described in SEQ ID NO:3 (whose gene sequence is represented by SEQ ID NO:4) via a linker (whose amino acid sequence and gene sequence are represented by SEQ ID NO:5 and SEQ ID NO:6, respectively).

The gene (SEQ ID NO:8) encoding the above polypeptide (SEQ ID NO:7) is obtained by overlapping PCR which is often performed to give a gene fragment. A BamHI restriction enzyme site is added to the 5′ terminus of the resulting amplified gene fragment, while an EcoRI restriction enzyme site is added to the 3′ terminus. The amplified gene fragment is inserted in-frame into the pGEX-6P-1 vector. Subsequently, the sequence is confirmed by a DNA sequencer.

The gene expression vector constructed as described above is used to transform Escherichia coli BL21. After performing incubation for 16 hours, a single colony is picked from the culture plate to inoculate 3 ml of 2×YT culture medium (tryptone; 16 wt %, yeast extract; 10 wt %, sodium chloride; 5 wt %, containing ampicillin at final concentration of 100 ug/ml). A shaking culture (preculture) is then performed at 37° C. After 12 hours, 3 ml of the culture is added to 250 ml of 2×YT culture medium (containing ampicillin at final concentration of 100 μg/ml) to perform a shaking culture at 28° C. When the absorbance OD₆₀₀ of the culture reaches 0.8, IPTG (isopropyl-β-D-galactopyranoside) is added to the culture at a final concentration of 1 mM to induce production of the polypeptide and incubated for 12 hours.

(1-2) Purification Process of the Fusion Polypeptide

The IPTG-induced E.coli cells are harvested (8000×g, 2 minutes, 4° C.) and resuspended in one tenth volume of phosphate buffered saline at 4° C. ((PBS) NaCl; 8 g, Na₂HPO₄; 1.44 g, KH₂PO₄; 0.24 g, KCl; 0.2 g, purified water; 1000 ml). The bacterial cells are ground by freeze-thawing and sonication, and solid impurities are removed by centrifugation (8000×g, 10 minutes, 4° C.). The GST fusion polypeptide whose expression is induced is purified with Glutathione Sepharose 4B (Amersham Biosciences Plc) by the manufacturer's recommended method. Glutathione sepharose to be used is subjected to a treatment to suppress the non-specific absorption in advance. More specifically, the glutathione sepharose is washed three times with an equal volume of PBS (8000×g, 1 minute, 4° C.). An equal volume of PBS containing 4% bovine serum albumin is then added thereto to treat for 1 hour at 4° C. After the treatment, the resulting glutathione sepharose is washed twice with an equal volume of PBS and resuspended in one-half volume of PBS. 40 μl of the pretreated glutathione sepharose is added to 1 ml of cell-free extract to gently agitate at 4° C. Through the aforementioned procedure, the GST fusion polypeptide is absorbed to the glutathione sepharose. After absorption, the glutathione sepharose is collected by centrifugation (8000×g, 1 minute, 4° C.), and washed three times with 400 μl of PBS. Subsequently, 40 μl of 10 mM glutathione is added thereto to agitate for 1 hour at 4° C. to allow the absorbed fusion polypeptide to elute. After the supernatant is collected by centrifugation (8000×g, 2 minutes, 4° C.), the glutathione is removed through dialysis against PBS to purify the GST fusion polypeptide. The band of the GST fusion polypeptide is confirmed by SDS-PAGE.

The GST fusion polypeptide is digested using PreScission protease (Amersham Pharmacia Biotech K. K, 5U) by the manufacturer's recommended method. Subsequently, the product is passed through the glutathione sepharose to remove the protease and GST. A 42 kDa band is confirmed by SDS-PAGE.

(2) Preparation of an Anti-HEL Antibody Fragment (HyHEL10 scFv)

A gene expression vector is constructed based on the pGEX-6P-1 (Amersham Biosciences Plc) vector to allow expression of the gene encoding a scFv peptide of HyHEL10 represented by SEQ ID NO:1. The scFv peptide of HyHEL10 is produced and purified by the same methods as described in the above 1-1 and 1-2.

(3) Preparation of an Anti-HEL Antibody Fragment (D1.3 scFv)

A gene expression vector is constructed based on the pGEX-6P-1 (Amersham Biosciences Plc) vector to allow expression of the gene (SEQ ID NO:10) encoding a scFv peptide of D1.3 represented by SEQ ID NO:9. The scfv peptide of D1.3 is produced and purified by the same methods as described in the above 1-1 and 1-2.

(4) Manufacture of a Biosensor Element

Subsequently, a sensor element including a primary antibody capturing HEL is manufactured.

In the present embodiment, a magnetoresistance effect element is used as a magnetic sensor. Therefore, the aforementioned detection region refers to the upper surface of the magnetoresistance effect element.

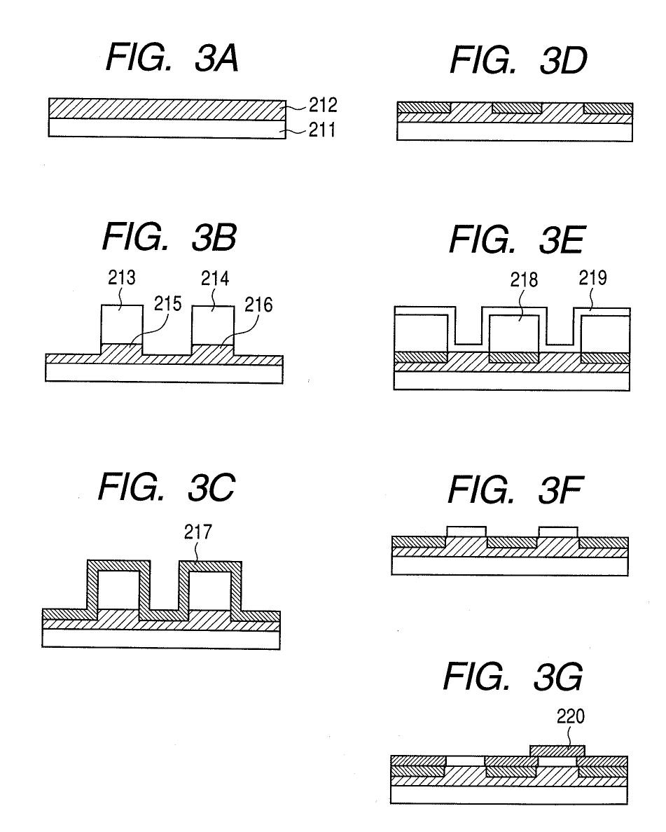

The magnetoresistance effect element of the present embodiment is manufactured through the following process. A magnetoresistive film 212 consisting of Ta (30 nm)/PtMn (20 nm)/CoFe (2 nm)/Ru (0.8 nm)/CoFe (2 nm)/AlOx (1.6 nm)/CoFe (3 nm)/Ru (5 nm)/Au (5 nm) is disposed on a silicon wafer 211 (FIG. 3A). Resist mask patterns 213 and 214 are formed in the region of a sensor element 215 and a reference element 216, and reactive ion etching is conducted to etch the surrounding of the sensor element 215 and the reference element 216. The sensor element 215 and the reference element 216 are in the identical configuration. Etching is controlled to stop at an AlOx film, and the metallic film below the AlOx film is kept to function as a lower electrode (FIG. 3B). After etching, an SiN insulating film (14 nm) 217 is disposed as an interlayer insulation film (FIG. 3C). The insulation film at the upper side of the sensor element 215 and the reference element 216 is polished by polishing. Subsequently, the resist mask patterns 213 and 214 are melted by a solvent to open the upper side of the sensor element 215 and the reference element 216 (FIG. 3D). A resist mask pattern 218 is formed to form an upper electrode. Then, an Au (20 nm) 219 is disposed (FIG. 3E). The unnecessary Au film and resist mask patterns are lifted-off using a solvent to form an upper electrode (FIG. 3F). In addition, SiN (20 nm) 220 is disposed after forming a resist mask pattern and a lift-off is performed in order to coat the surface of the electrode except the upper side of the sensor element with a non-immobilized film (FIG. 3G).

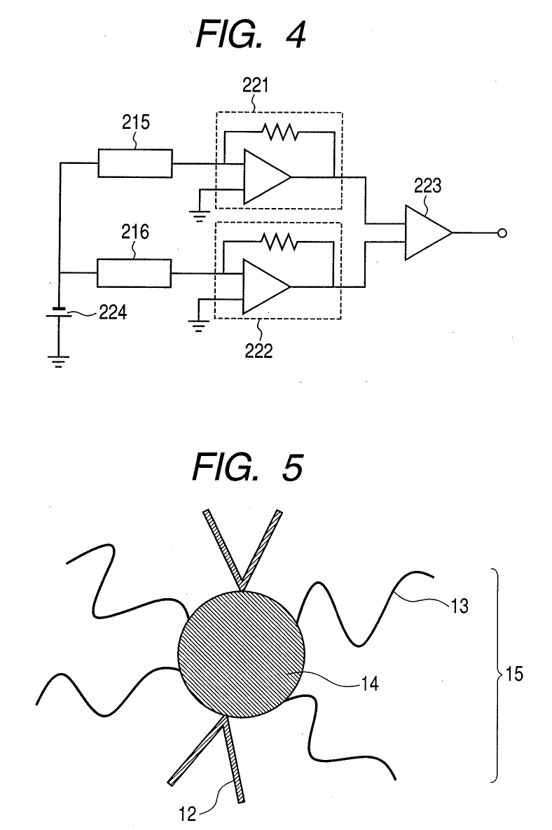

The sensor element 215 and the reference element 216 are electronically connected in parallel so that a voltage equal in magnitude is applied. The current flowing in the sensor element 215 and current flowing in the reference element 216 are converted to voltage levels by I/V converters 221 and 222, and the difference in the voltages is output by a differential amplifier 223 to detect the presence and number of the antigen (target substance) (see FIG. 4).

Although a biosensor device is constructed from one sensor element in the present embodiment, the present invention also includes embodiments wherein a plurality of sensor elements is installed. In such cases, detection signals of each sensor element can be obtained by sequentially switching sensor elements with a selection transistor to enable detection of a large number of antigens (target substances) or a wide variety of antigens (target substances).

As a first capture agent, the scfv of D1.3 prepared as a primary antibody capturing HEL in the above 3 is immobilized to the surface of the sensor element 215 manufactured by the method described above. First, an ethanol solution of 10-carboxy-1-decanethiol is applied to the detection region. Through this operation, a carboxyl group is exposed to the surface of the Au film. Subsequently, an N-hydroxy sulfosuccinimide aqueous solution and a 1-ethyl-3-(3-dimethlaminopropyl)carbodiimide hydrochloride aqueous solution are applied by the same method. Through these operations, a succinimide group is exposed to the surface of the Au film. The succinimide group and an amino group of the D1.3 scFv are reacted to enable immobilization of the primary antibody fragment D1.3 scFv capturing HEL as the first capture agent. In addition, the unreacted succinimide group on the surface of the Au film may be detached by adding hydroxylamine chloride.

(5) Agglutination Experiment of a Magnetic Marker

The fusion polypeptide prepared in the aforementioned 1 and a magnetic marker particle (nanomag-D-spio, NH₂ modified, particle diameter 50 nm; Corefront corp.) are used to perform the following operations to enable agglutination of the magnetic marker: 1) Dissolve the fusion polypeptide in a phosphate buffer (pH 7.0); and 2) Add the magnetic marker to the phosphate buffer containing the fusion polypeptide.

After performing the above operations, agglutination of ferrite can be confirmed by incubating the mixture for a few minutes at room temperature. Agglutination of the magnetic marker can be confirmed by direct observation such as dynamic light scattering method and TEM.

(6) Control Agglutination Experiment of the Magnetic Marker

The scFv of HyHEL10 prepared in the aforementioned 2 and the magnetic marker particle used in the aforementioned 5 can be used to conduct the same experiment as in the aforementioned 5 in order to confirm that agglutination of ferrite does not occur.

(7) HEL Detection

The capture agent member and biosensor element manufactured in the above 1 through 4 as well as the magnetic marker used in the above 5 can be used to perform the following operations in an attempt to detect HEL: 1) Immerse the above sensor element in a phosphate buffer containing HEL serving as the antigen (target substance); 2) Wash the unreacted HEL with a phosphate buffer; 3) Immerse the above biosensor element which completed Processes 1) and 2) in a phosphate buffer containing the capture agent member to which a labeling agent is immobilized; 4) Wash the unreacted capture agent member with a phosphate buffer; 5) Add a magnetic marker; and 6) Wash the unreacted magnetic marker with a phosphate buffer.

Through the above operations, the target substance is captured by the primary antibody and the secondary antibody fragment, and the magnetic marker is immobilized to the sensor as illustrated in FIG. 2. More specifically, when the target substance is not present in the test solution, the capture agent member is not retained in the sensor, and the magnetic marker does not agglutinate to the sensor. Therefore, the target substance can be detected by detecting the presence of the magnetic marker. In addition, the amount of the target substance contained in the test solution can be indirectly revealed by detecting the number of the magnetic marker immobilized. Improvement of detection sensitivity of the magnetic sensor can be achieved by allowing the magnetic marker to selectively agglutinate using the capture agent member of the present embodiment.

### Example 2

<<Detection Using Agglutination of a Magnetic by Molecular Recognition>>

Evaluation is conducted by the same method as in Example 1, except that the capture agent member and the magnetic marker of Example 1 are replaced with the capture agent member and a silica particle containing magnetite (nanomag-silica (particle diameter 130 nm), Corefront corp.) used as the magnetic marker of the present embodiment.

(1) Preparation of an Anti-HEL Antibody Fragment (scfv) Labeled With a Silicon Dioxide Specific Peptide

A fusion polypeptide (SEQ ID NO:13) of a silicon dioxide specific peptide represented by SEQ ID NO:11 (whose gene sequence is represented by SEQ ID NO:12) and HyHEL10 scfv is produced and purified by the same method as in Example 1. The gene sequence of the fusion polypeptide is represented by SEQ ID NO:14.

### Example 3

<<Detection Using Agglutination of a Magnetic by a Stimuli Responsive Polymer>>

(1) Manufacture of a Ferrite Particle Coated With a Stimuli Responsive Polymer

(1-1) Synthesis of Magnetite (10 nm)

After FeCl₃ and FeCl₂4H₂ are dissolved in distilled water to form an aqueous solution, nitrogen bubbling is performed for 30 minutes. Subsequently, while the resulting aqueous solution is vigorously agitated with a stirrer, 29% aqueous ammonia is added to synthesize Magnetite 1. The synthesized Magnetite 1 is purified by dialysis, and preserved in powder state by freeze drying. TEM is performed to evaluate the particle diameter of Magnetite 1, and it is confirmed that the particle is 10 nm in size and that it has a uniform particle diameter distribution.

(1-2) Introduction of a Polymerization Initiation Group to the Magnetite Surface

((Chloromethyl)phenylethyl)trichlorosilane is dissolved in anhydrous toluene, and the powdered Magnetite 1 is added to the solution. The mixture is subjected to ultrasonic treatment under agitation conditions at 4° C. to disperse Magnetite 1 in the solution. The dispersion is left to stand for 2 hours while being subjected to ultrasonic treatment under agitation conditions at room temperature to synthesize Magnetite 2, wherein a chloromethyl group derived from ((Chloromethyl)phenylethyl)trichlorosilane is introduced to the surface of Magnetite 1. Magnetite 2 is purified by dialysis, and preserved in powder state after drying.

Subsequently, sodium N,N-diethyldithiocarbamate is dissolved in distilled water, and the powdered Magnetite 2 is added to the solution. The mixture is subjected to ultrasonic treatment under agitation conditions at room temperature to disperse Magnetite 2 in the solution. The dispersion is left to stand for 2 hours while being subjected to ultrasonic treatment under agitation conditions at room temperature to synthesize Magnetite 3, wherein N,N-diethyldithiocarbamate is introduced to the surface of Magnetite 2. Magnetite 3 is purified by dialysis, and preserved as an aqueous dispersion after being concentrated to a certain concentration with an evaporator.

(1-3) Polymer Coating of the Magnetite Surface

N-isopropyl acrylamide and acrylic acid are dissolved in distilled water to form a solution. Subsequently, an aqueous dispersion of the aforementioned Magnetite 3 is added to the solution to form a polymerization solution, and then nitrogen bubbling is performed for 30 minutes. Next, ultraviolet rays whose irradiation wavelengths are in the range of 312 nm to 577 nm are irradiated to the polymerization solution to perform the photo polymerization. As a result, Magnetite 4 is synthesized, wherein a copolymer constituting of N-isopropyl acrylamide and acrylic acid is included on the surface of Magnetite 3. Magnetite 4 is purified by dialysis, and the agglomerate is further removed by using a filter. Subsequently, Magnetite 4 is preserved as an aqueous dispersion after being concentrated to a certain concentration with an evaporator. When the particle diameter of Magnetite 4 is evaluated in water by dynamic light scattering method, it is confirmed that the particle is 32 nm in size and is excellent in size uniformity. In addition, TEM is performed to evaluate Magnetite 4, and it is confirmed that it has a core-shell type structure wherein the core includes magnetite and the shell includes a copolymer constituting of N-isopropyl acrylamide and acrylic acid.

(2) Preparation of an Anti-HEL Antibody Fragment (scFv) Labeled With a Stimuli Responsive Polymer

An N-hydroxy sulfosuccinimide aqueous solution and 1-ethyl-3-(3-dimethlaminopropyl)carbodiimide hydrochloride aqueous solution are added to Magnetite 4 prepared in the aforementioned 1-3 to activate a carboxy group. Subsequently, an amino group of the anti-HEL antibody fragment prepared in 3 of Example 1 is reacted thereto to give an anti-HEL antibody fragment labeled with a stimuli responsive polymer.

(3) Agglutination Experiment of a Magnetic Marker

The anti-HEL antibody fragment labeled with a stimuli responsive polymer and the ferrite particle coated with a stimuli responsive polymer prepared in the aforementioned 1 and 2 are used to perform the following operations to enable agglutination of the magnetic marker: 1) Dissolve the anti-HEL antibody fragment labeled with a stimuli responsive polymer in a phosphate buffer (pH 7.0) at 28° C., which is lower than or equal to the lower critical temperature; 2) Add the ferrite particle coated with a stimuli responsive polymer to the aforementioned solution, while maintaining the temperature at 28° C.; and 3) Bring up the solution temperature to 35° C., which is equal to or higher than the critical temperature.

After performing the above operations, agglutination of ferrite can be confirmed by incubating the mixture for a few minutes at 35° C. Agglutination of the magnetic marker can be confirmed by direct observation such as dynamic light scattering method and TEM.

### Example 4

<<Utilization of a Branched Labeling Agent>>

Evaluation is conducted by the same method as in Example 1, except that the capture agent member and the magnetic marker of Example 1 are replaced with the capture agent member and a silica particle containing magnetite (nanomag-silica (particle diameter 130 nm), Corefront corp.) used as the magnetic marker of the present embodiment.

(1) Preparation of a Capture Agent Member

A polypeptide (SEQ ID NO:15) fused by repeating 10 times the silicon dioxide specific peptide (SEQ ID NO:11) is produced and purified by the same method as in Example 1. The gene sequence of the polypeptide is represented by SEQ ID NO:16.

An N-hydroxy sulfosuccinimide aqueous solution and 1-ethyl-3-(3-dimethlaminopropyl)carbodiimide hydrochloride aqueous solution are added to a solution of the magnetic particle (Magnetite 4) prepared in 1-3 of Example 3 to expose an activated succinimide group on the surface of the magnetic particle. An amino group of the polypeptide represented by SEQ ID NO:15 is reacted with the succinimide group to immobilize the silicon dioxide specific polypeptide to the surface of the magnetic particle. Subsequently, the HyHEL10 scfv prepared in 3 of Example 1 is added and immobilized to the surface of the magnetic particle by the same method to prepare the capture agent member of the present invention (FIG. 5). In addition, the unreacted succinimide group on the surface of the magnetic particle may be detached by adding hydroxylamine chloride.

### Example 5

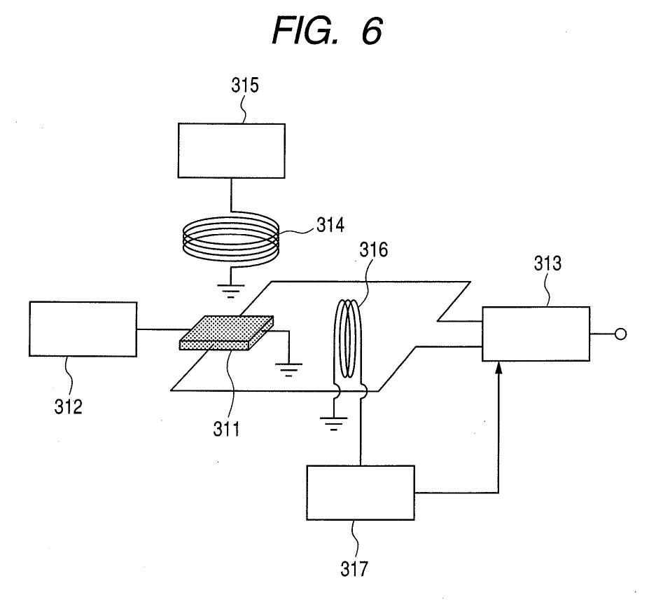

Evaluation is conducted by the same method as in Example 1 by replacing the detection system from a magnetoresistance effect element of Example 1 to a Hall effect element. The target substance can be detected with high sensitivity by utilizing agglutination of the magnetic marker by a biosensor element of the present embodiment. A Hall effect element 311 consisting of InSb is formed on a GaAs substrate by using a semiconductor process. A DC power supply 312 for applying a detection current and a detection unit for detecting the Hall electromotive force are connected to the Hall effect element 311. A lock-in amplifier 313 is used as a detection means. At the time of detection, a coil 314 and a DC power supply 315 are used to apply a DC magnetic field vertically to the film surface of the Hall effect element 311, wherein the DC magnetic field is of a magnitude enough for saturating magnetization of the magnetic marker. Simultaneously, a coil 316 and an AC power supply 317 are used to apply an AC magnetic field which is of a comparably small magnitude in a film surface inward direction of the Hall effect element 311. By doing this, the magnetization of the magnetic marker oscillates from side to side while maintaining the magnitude thereof. By oscillating the magnetization from side to side, the film surface vertical component of the floating magnetic field originating from the magnetic marker oscillates at a frequency twice as high as that of the AC magnetic field. Therefore, signals associated with the presence and number of the magnetic marker can be detected by only extracting the component whose frequency is twice as high as that of the AC magnetic field from the voltage of the Hall effect element 311 by the lock-in amplifier 313 (see FIG. 6).

### Example 6

Evaluation is conducted by the same method as in Example 2 by replacing the detection system from a magnetoresistance effect element of Example 2 to a Hall effect element. The target substance can be detected with high sensitivity by utilizing agglutination of the magnetic marker by a biosensor element of the present embodiment.

### Example 7

Evaluation is conducted by the same method as in Example 3 by replacing the detection system from a magnetoresistance effect element of Example 3 to a Hall effect element. The target substance can be detected with high sensitivity by utilizing agglutination of the magnetic marker by a biosensor element of the present embodiment.

### Example 8

Evaluation is conducted by the same method as in Example 4 by replacing the detection system from a magnetoresistance effect element of Example 4 to a Hall effect element. The target substance can be detected with high sensitivity by utilizing agglutination of the magnetic marker by a biosensor element of the present embodiment.

### Example 9

Evaluation is conducted by the same method as in Example 1, except that the detection system is replaced from a magnetoresistance effect element of Example 1 to a superconducting quantum interference device element, and that micromer-M (particle diameter ranging from 2 to 12 nm) having a surface modified with NH₂ manufactured by Corefront corp. is used as the magnetic marker. The target substance can be detected with high sensitivity by utilizing agglutination of the magnetic marker by a biosensor element of the present embodiment.

### Example 10

Evaluation is conducted by the same method as in Example 2 by replacing the detection system from a magnetoresistance effect element of Example 2 to a superconducting quantum interference device element. The target substance can be detected with high sensitivity by utilizing agglutination of the magnetic marker by a biosensor element of the present embodiment.

### Example 11

Evaluation is conducted by the same method as in Example 3 by replacing the detection system from a magnetoresistance effect element of Example 3 to a superconducting quantum interference device element. The target substance can be detected with high sensitivity by utilizing agglutination of the magnetic marker by a biosensor element of the present embodiment.

### Example 12

Evaluation is conducted by the same method as in Example 4 by replacing the detection system from a magnetoresistance effect element of Example 4 to a superconducting quantum interference device element. The target substance can be detected with high sensitivity by utilizing agglutination of the magnetic marker by a biosensor element of the present embodiment.

### Example 13

Evaluation is conducted by the same method as in Example 1 by replacing the detection system from a magnetoresistance effect element of Example 1 to a magnetic impedance element. The target substance can be detected with high sensitivity by utilizing agglutination of the magnetic marker by a biosensor element of the present embodiment.

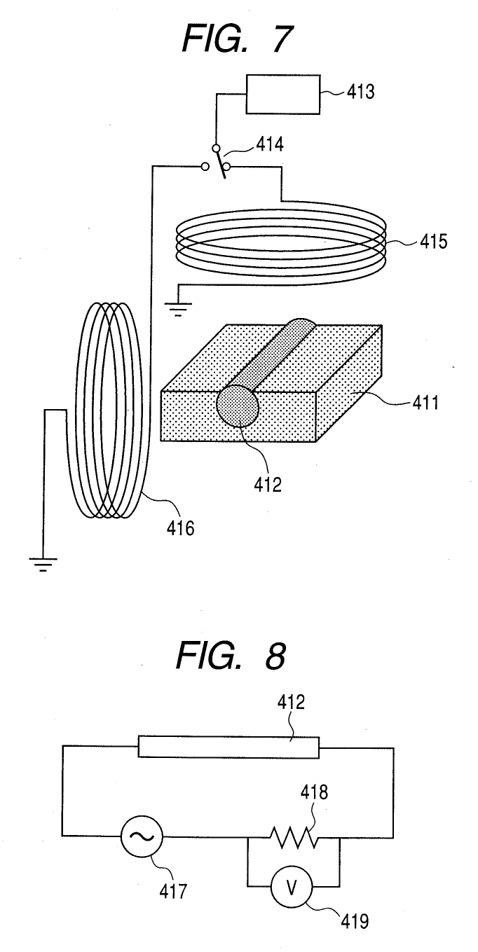

A magnetic impedance element 412 consisting of amorphous CoFeSiB is formed embedded within a substrate 411 consisting of an insulator as illustrated in FIG. 7. The magnetic marker is not immobilized to the substrate 411, and is only immobilized to the upper side of the magnetic impedance element 412, where the upper side is uncoated with the substrate 411. A power supply 413, a switching switch 414, coils 415 and 416 are installed to apply a magnetic field vertically to the longitudinal direction of the magnetic impedance element.

As illustrated in FIG. 8, an AC power supply 417 for applying a high-frequency current of 10 MHz and a fixed resistor 418 are connected in series to the magnetic impedance element 412. A voltmeter 419 is connected to the fixed resistor 418 in order to measure the voltage.

The direction of a magnetic field applied to the magnetic impedance element is changed by alternately applying a current equal in magnitude to the coils 415 and 416 in order to detect the magnetic marker. When the direction of the applied magnetic field is altered, the magnitude of the floating magnetic field originating from the magnetic marker changes in the vicinity of the surface of the magnetic impedance element. This results in changes in impedance of the magnetic impedance element. By analyzing the variation of this change, the presence and number of the magnetic marker can be detected.

### Example 14

Evaluation is conducted by the same method as in Example 2 by replacing the detection system from a magnetoresistance effect element of Example 2 to a magnetic impedance element. The target substance can be detected with high sensitivity by utilizing agglutination of the magnetic marker by a biosensor element of the present embodiment.

### Example 15

Evaluation is conducted by the same method as in Example 3 by replacing the detection system from a magnetoresistance effect element of Example 3 to a magnetic impedance element. The target substance can be detected with high sensitivity by utilizing agglutination of the magnetic marker by a biosensor element of the present embodiment.

### Example 16

Evaluation is conducted by the same method as in Example 4 by replacing the detection system from a magnetoresistance effect element of Example 4 to a magnetic impedance element. The target substance can be detected with high sensitivity by utilizing agglutination of the magnetic marker by a biosensor element of the present embodiment.

### Example 17

<<Labeling for Improving Quantitative Performance by Regulating the Number of the Agglutinating Magnetic Marker>>

Evaluation is conducted by the same method as in Example 1, except that the capture agent member and the magnetic marker of Example 1 are replaced with the capture agent member and a magnetic particle modified with biotin (nanomag-silica (particle diameter 250 nm), Corefront corp.) used as the magnetic marker of the present embodiment.

(1) Preparation of a Capture Agent Member

A polypeptide (SEQ ID NO:19) wherein streptavidin (SEQ ID NO:17; corresponding gene, SEQ ID NO:18) is fused is produced and purified by the same method as in Example 1. The gene sequence of the polypeptide is represented by SEQ ID NO:20.

(2) Detection Experiment

The number of the magnetic marker agglutinating to a secondary antibody fragment bound to the target substance which is captured to the sensor element is 1 per secondary antibody fragment molecule. As a result, the concentration of the target substance correlates with the number of the magnetic marker being immobilized to the sensor element, leading to a quantitative evaluation.

### Example 18

Evaluation is conducted by the same method as in Example 17 by replacing the detection system from a magnetoresistance effect element of Example 17 to a Hall effect element. The target substance can be quantitatively detected by utilizing agglutination of the magnetic marker by a biosensor element of the present embodiment.

### Example 19

Evaluation is conducted by the same method as in Example 17 by replacing the detection system from a magnetoresistance effect element of Example 17 to a superconducting quantum interference device element. The target substance can be quantitatively detected by utilizing agglutination of the magnetic marker by a biosensor element of the present embodiment.

### Example 20

Evaluation is conducted by the same method as in Example 17 by replacing the detection system from a magnetoresistance effect element of Example 17 to a magnetic impedance element. The target substance can be quantitatively detected by utilizing agglutination of the magnetic marker by a biosensor element of the present embodiment.

According to the exemplary embodiments of the present invention described above, reactivity and dispersibility of a secondary capture agent in a sandwich method can be favorably maintained, and detection sensitivity for a magnetic marker can be improved by allowing the magnetic particle to agglutinate with a nucleus of a labeling agent immobilized to the second capture agent after the second capture agent captures the target substance.

In addition, according to the exemplary embodiments of the present invention, a magnetic particle can agglutinate selectively to the labeling agent which is used to label the secondary capture agent member reacted with the captured agent by labeling the capture agent with a labeling agent serving as an agglutination nucleus for the magnetic particle. This enables improvement in detection sensitivity.

According to these, there is provided a biosensor enabling detection of the target substance with high sensitivity.

While the present invention has been described with reference to exemplary embodiments, it is to be understood that the invention is not limited to the disclosed exemplary embodiments and various changes may be freely made in materials, composition conditions and reaction conditions without departing from the range in which a biosensor having the same functions and effects can be obtained. The scope of the following claims is to be accorded the broadest interpretation so as to encompass all such modifications and equivalent structures and functions.

This application claims the benefit of Japanese Patent Application No. 2007-081942, filed Mar. 27, 2007, and Japanese Patent Application No. 2008-057728, filed Mar. 7, 2008, which are hereby incorporated by reference herein in their entirety.

What is claimed is:

1. A capture agent member being used in a magnetic sensor to detect the presence or concentration of a target substance in a test solution by detecting the presence or number of a magnetic marker, wherein the capture agent member comprises a capture agent for capturing the target substance and a labeling agent serving as a nucleus for the magnetic marker to agglutinate, and wherein the capture agent is labeled with the labeling agent.

2. The capture agent member according to claim 1, wherein the labeling agent has a surface charge in the test solution.

3. The capture agent member according to claim 2, wherein the labeling agent has a surface charge whose polarity differs from the magnetic marker.

4. The capture agent member according to claim 1, wherein the labeling agent is a stimuli responsive polymer.

5. The capture agent member according to claim 4, wherein the stimuli responsive polymer is at least partially contained in the magnetic marker.

6. The capture agent member according to claim 1, wherein the labeling agent is a material specific peptide.

7. The capture agent member according to claim 6, wherein the material specific peptide has affinity for a material at least partially contained in the magnetic marker.

8. A method for detecting the presence or concentration of the target substance in a test solution by detecting the presence or number of the magnetic marker, wherein the detection method comprises reacting a first capture agent which captures the target substance in the test solution with the target substance, reacting a capture agent member comprising a second capture agent which captures the target substance with the target substance being reacted with the first capture agent, and causing agglutination of the magnetic marker with a nucleus of a labeling agent used to at least partially label the capture agent member comprising the second capture agent.

9. The detection method according to claim 8, wherein causing the magnetic marker to agglutinate is induced by electrostatic interaction.

10. The detection method according to claim 8, wherein causing the magnetic marker to agglutinate is induced by molecular recognition.

11. The detection method according to claim 8, wherein causing the magnetic marker to agglutinate is induced by an external stimulus.

12. The detection method according to claim 8, wherein the method further comprises washing with a solution at least once between the reaction and agglutination.

13. A kit being used in a magnetic sensor to detect the presence or concentration of a target substance in the test solution by detecting the presence or number of the magnetic marker, wherein the kit comprises a sensor element, a capture agent member and a magnetic marker, wherein the sensor element comprises a sensor element member and a first capture agent being immobilized to the surface of the member and capturing the target substance, the capture agent member comprises a second capture agent for capturing the target substance and a labeling agent having a surface charge in the test solution, and the magnetic marker comprises a magnetic material having a charge whose polarity differs from that of the labeling agent, or comprises a magnetic material and a material having a charge whose polarity differs from the labeling agent.

14. A kit being used in a magnetic sensor to detect the presence or concentration of a target substance in the test solution by detecting the presence or number of the magnetic marker, wherein the kit comprises a sensor element, a capture agent member and a magnetic marker, wherein the sensor element comprises a sensor element member and a first capture agent being immobilized to the surface of the member and capturing the target substance, the capture agent member comprises a second capture agent for capturing the target substance and a labeling agent at least partially comprising a material specific peptide, and the magnetic marker comprises a magnetic material and a material having affinity for the material specific peptide.

15. A kit being used in a magnetic sensor to detect the presence or concentration of a target substance in the test solution by detecting the presence or number of the magnetic marker, wherein the kit comprises a sensor element, a capture agent member and a magnetic marker, wherein the sensor element comprises a sensor element member and a first capture agent being immobilized to the surface of the member and capturing the target substance, the capture agent member comprises a second capture agent for capturing the target substance and a labeling agent at least partially comprising a stimuli responsive polymer, and the magnetic marker comprises a magnetic material and the stimuli responsive polymer.
